(19) United States
(12) Patent Application Publication
(10) Pub. No.: US 2024/0151735 A1
(43) Pub. Date: May 9, 2024

(54) METHODS OF DETECTING ADVANCED LIVER FIBROSIS OR HEPATOCELLULAR CARCINOMA BIOMARKERS IN A SAMPLE

(71) Applicant: BOARD OF REGENTS, THE UNIVERSITY OF TEXAS SYSTEM, Austin, TX (US)

(72) Inventor: Laura BERETTA, Bellaire, TX (US)

(21) Appl. No.: 18/548,762

(22) PCT Filed: Mar. 1, 2022
(86) PCT No.: PCT/US2022/018316

§ 371 Date: Sep. 1, 2023

Related U.S. Application Data
(60) Provisional application No. 63/155,155, filed on Mar. 1, 2021.

(51) Int. Cl. G01N 33/92; G01N 30/72

(52) U.S. Cl. CPC G01N 33/92; G01N 30/72; G01N 2405/00; G01N 2800/085

(57) ABSTRACT

A method of detecting hepatocellular carcinoma (HCC) biomarkers in a sample from a human subject by detecting hepatocellular carcinoma biomarkers in the sample. The HCC biomarkers can include Tetracosanoic Acid, Heptadecanoic Acid, Eicosapentaenoic Acid, or Docosapentaenoic Acid. Optionally, the HCC biomarkers can further include osteopontin (OPN) and/or alpha-fetoprotein (AFP). The sample is a sample of a bodily fluid obtained from a human subject. The subject can be a healthy patient, a patient suspected of having a liver disorder, a patient previously diagnosed with liver cirrhosis, or a patient previously diagnosed with liver fibrosis.

### CROSS-REFERENCE TO RELATED APPLICATIONS

This application claims priority to and the benefit of U.S. Provisional Patent Application Ser. No. 63/155,155, filed Mar. 1, 2021, the contents of which is incorporated herein by this reference as if fully set forth herein.

### GOVERNMENT LICENSE RIGHTS

This invention was made with government support under grant numbers CA 217674 and CA 195524 awarded by the National Institutes of Health. The government has certain rights in the invention.

### FIELD

The present application relates to the field of cancer marker detection and particularly relates to the detection of hepatocellular carcinoma biomarkers.

### BACKGROUND

Liver cancer is a lethal malignancy and a heavy disease burden globally with one million cases and 829,000 deaths in 2016. Hepatocellular carcinoma (HCC) is the predominant form of primary liver cancer and is restricted largely to patients with cirrhosis or advanced fibrosis. HCC incidence has increased over the past years in Western Europe and the United States. HCC has the second lowest five-year survival (18%), trailing only pancreatic cancer. This is largely due to the fact that the majority of HCC cases are diagnosed at advanced stages when curative therapy is impossible. The degree of liver fibrosis is the strongest predictive factor for life-threatening complications including HCC.

Non-alcoholic fatty liver disease (NAFLD) constitutes an increasingly important risk for hepatocellular carcinoma (HCC). Because of the epidemics of obesity and type 2 diabetes, the prevalence of NAFLD has increased steadily to reach global prevalence of 25%. In patients with NAFLD, the degree of liver fibrosis is the strongest predictive factor for life-threating complications including HCC. It is therefore of utmost importance to detect early stage liver fibrosis and prevent its progression.

In the United States, Hispanics have the highest prevalence of NAFLD and Hispanics in South Texas have the highest age-adjusted rate of HCC. The Cameron County Hispanic Cohort (CCHC) is a population-based cohort of Hispanics in South Texas at the US-Mexico border region, with high prevalence of obesity (51%), diabetes (28%) and NAFLD (49%). Liver cancer ranked third among cancers in males and sixth in females based on self-reported data. A four-fold higher prevalence of advanced liver fibrosis and cirrhosis in this population has also been reported compared to the general US population, primarily attributable to central obesity and diabetes.

To reduce HCC-related mortality, early detection of small tumors that are amenable to curative treatments is urgently needed. Because such tumors are usually asymptomatic, novel biomarkers for identification of high-risk individuals who would benefit from surveillance, as well as those for early detection of this highly lethal cancer are needed. The major risk factor for developing HCC is liver cirrhosis resulting from chronic hepatitis B virus (HBV), hepatitis C virus (HCV), alcohol abuse, or nonalcoholic steatohepatitis (NASH). Current guidelines recommend surveillance for HCC in patients with cirrhosis of any etiology.

For the last 40 years, α-fetoprotein (AFP) has been the only serum biomarker used by clinicians together with liver ultrasound for the surveillance of HCC, despite its low sensitivity for the detection of early-stage HCC. In addition to AFP, FDA-approved markers for HCC surveillance are the fucosylated isoform of AFP (AFP-L3) and des-gamma-carboxy-prothrombin (DCP). However, these markers are not included in HCC surveillance guidelines and their clinical utility requires further evaluation.

Therefore, novel biomarkers for HCC surveillance in patients, particularly in cirrhotic patients and in patients with liver fibrosis, and methods for the detection of those markers are urgently needed.

### BRIEF SUMMARY

A method of detecting hepatocellular carcinoma (HCC) biomarkers in a sample is provided herein by detecting in the sample one or more (e.g., two or more) hepatocellular carcinoma biomarkers. The two or more HCC biomarkers are select fatty acids including Tetracosanoic Acid, Heptadecanoic Acid, Eicosapentaenoic Acid, or Docosapentaenoic Acid, and the sample is a sample of a bodily fluid obtained from a human subject. Additional or alternative HCC biomarkers to be detected in accordance with the method include 22:6n3-Docosahexaenoic acid, 20:0-Eicosanoic acid, 23:0 Tricosanoic acid, and 25:0-Pentacosanoic acid. Other additional or alternative HCC biomarkers to be detected in accordance with the method are further listed in the Detailed Description of this disclosure.

Optionally, the two or more hepatocellular carcinoma biomarkers further include osteopontin (OPN) and/or alpha-fetoprotein (AFP).

The quantity of HCC biomarkers in the sample can be quantified to determine whether a threshold of increase or decrease has been achieved that provides an indication of the progression or regression of disease.

In one embodiment, the subject is a patient previously diagnosed with liver cirrhosis. In another embodiment, the subject is a patient previously diagnosed with liver fibrosis.

In accordance with the method, free fatty acids present in the sample are detected by gas chromatography (GC) or liquid chromatography (LC) and/or mass spectrometry (MS or mass spec). In one embodiment, samples are prepared for detection by the following procedures or equivalents thereof: extracting free fatty acids from the sample, converting the free fatty acids to acyl chloride intermediates, derivatizing the acyl chloride intermediates to form derivatized products, and analyzing the derivatized products gas chromatography, by gas chromatography-mass spec or by liquid chromatography-mass spectrometry (LC-MS) to detect the two or more fatty acid HCC biomarkers.

In one embodiment, the detecting comprises determining the concentrations of each of the two or more biomarkers in the sample.

The bodily fluid from which the sample is obtained is preferably plasma, but can also be blood, sera or any other bodily fluid that contains fatty acids.

In some embodiments, the subject may be at risk of developing HCC or advanced liver fibrosis based on the risk factors described in the Examples of this disclosure. In some instances, the subject may be determined to be at risk of developing HCC or advanced liver fibrosis based on the methods provided herein. In some instances, where the subject is determined to be at risk of developing HCC or advanced liver fibrosis based on the provided methods, the subject may be monitored for development of these conditions and/or may be treated for HCC or advanced liver fibrosis using one or more of the treatment methods described in this disclosure.

In some embodiments, the levels of the one or more (e.g., two or more) hepatocellular carcinoma biomarkers in the sample are compared to a control level, wherein the control level corresponds to the concentration of the two or more hepatocellular carcinoma biomarkers in a sample from a healthy individual without hepatocellular carcinoma.

In one embodiment, if the subject currently undergoing treatment for HCC, at least two samples can be obtained at different time points during the treatment and one of at least one of the different time points is a time point prior to start of that treatment.

If the subject is currently undergoing treatment for HCC, in one embodiment, the treatment is one or more of a drug treatment, a radiation treatment or a surgical treatment. In another embodiment, after detection of the hepatocellular carcinoma biomarkers, the patient is treated with or continues treatment with an anti-hepatocellular carcinoma treatment such as a drug treatment, a radiation treatment or a surgical treatment.

Also provided is a method of generating a report containing information on results of the detection of hepatocellular carcinoma biomarkers in a sample, including detecting two or more hepatocellular carcinoma biomarkers in the sample, and generating the report, wherein the two or more fatty acid HCC biomarkers are select fatty acids including Tetracosanoic Acid, Heptadecanoic Acid, Eicosapentaenoic Acid, or Docosapentaenoic Acid; the sample is a sample of a bodily fluid obtained from a subject, and the report is useful for diagnosing hepatocellular carcinoma in the subject. Additional or alternative HCC biomarkers to be detected in accordance with the method include 22:6n3-Docosahexaenoic acid, 20:0-Eicosanoic acid, 23:0 Tricosanoic acid, and 25:0-Pentacosanoic acid. The quantity of HCC biomarkers in the sample can be quantified to determine whether a threshold of increase or decrease has been achieved that provides an indication of the progression or regression of disease.

Also provided is a system for detecting hepatocellular carcinoma biomarkers in a sample, by utilizing a station for analyzing the sample by mass spectrometry (Mass Spec or MS) or liquid chromatography/mass spectrometry (LC/MS) to detect two or more hepatocellular carcinoma biomarkers in the sample, wherein the two or more fatty acid HCC biomarkers are select fatty acids including Tetracosanoic Acid; Heptadecanoic Acid, Eicosapentaenoic Acid, or Docosapentaenoic Acid, and the sample is a sample of a bodily fluid obtained from a subject. Optionally, a station for generating a report containing information on results of the analyzing is further included. Additional or alternative HCC biomarkers to be detected in accordance with the method include 22:6n3-Docosahexaenoic acid, 20:0-Eicosanoic acid, 23:0 Tricosanoic acid, and 25:0-Pentacosanoic acid. The quantity of HCC biomarkers in the sample can be quantified to determine whether a threshold of increase or decrease has been achieved that provides an indication of the progression or regression of disease.

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. The present document describes and refers to various embodiments of the invention. No particular embodiment is intended to define the scope of the invention. Rather, the embodiments merely provide non-limiting examples of various methods, that are at least included within the scope of the invention. Some embodiments of the present invention are summarized below, while others are described and shown elsewhere in the present document.

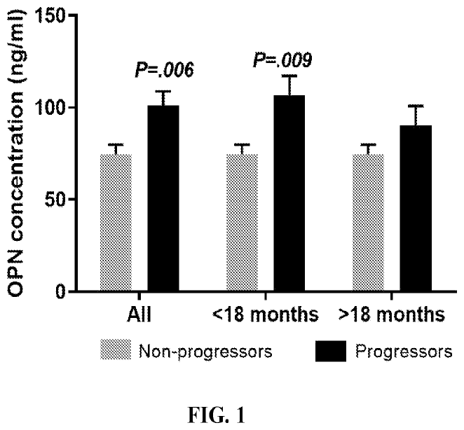

FIG. 1 shows concentrations of OPN in serum samples of cirrhotic patients who did not develop HCC during follow-up (non-progressors) and of cirrhotic patients with incident HCC during follow-up (progressors) according to aspects of this disclosure. The data are presented for all patients (All), for those HCC incidence within 18 months of follow-up (<18 m), and for those HCC incidence occurring after 18 months of follow-up (>18 m). Data are shown as mean+SEM. OPN, osteopontin.

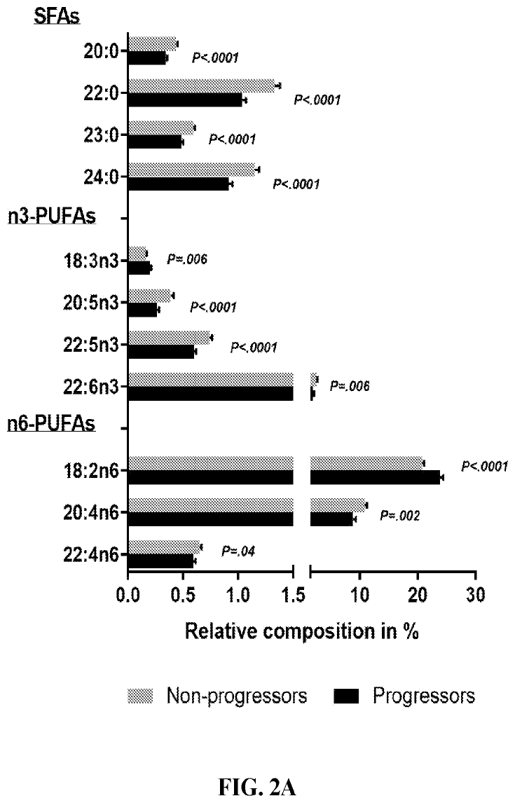

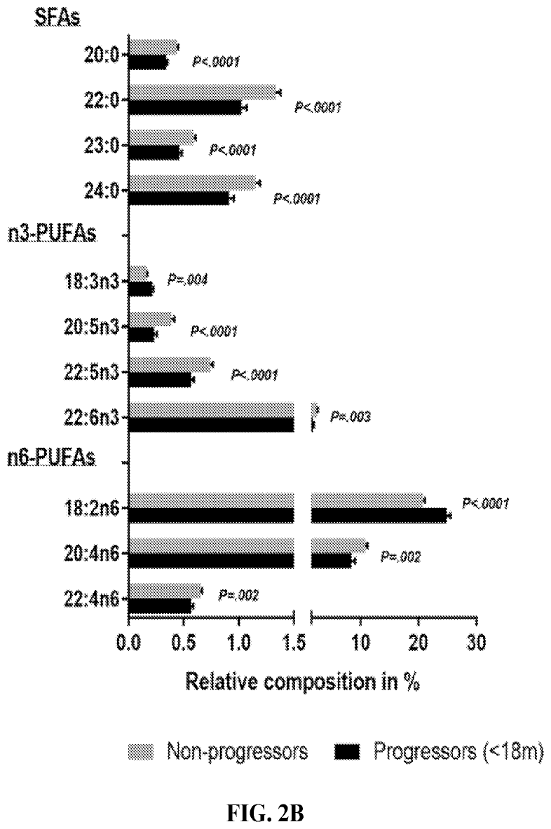

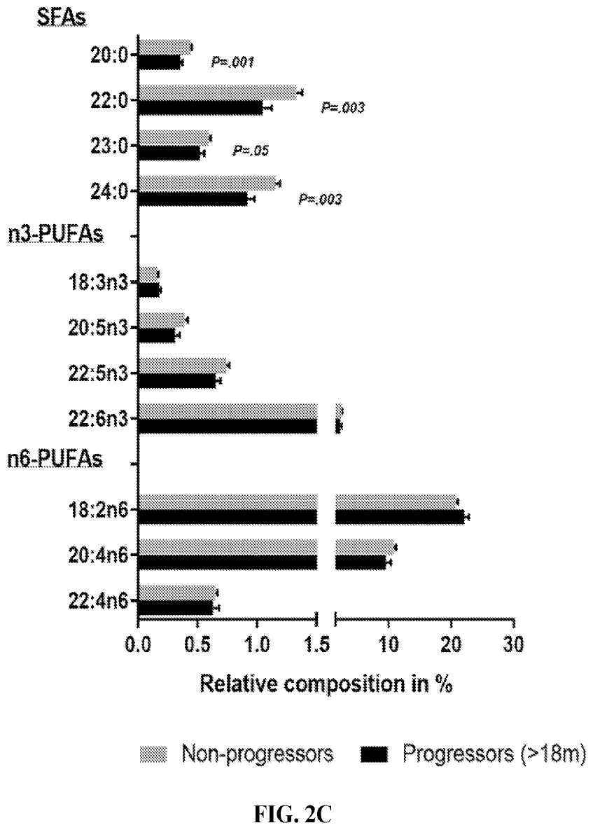

FIG. 2 shows FA levels in the 103 cirrhotic study participants according to aspects of this disclosure. Comparisons are shown between: (Panel A) Non-progressors (n=63) and progressors (n=40); (Panel B) Non-progressors (n=63) and progressors with a diagnosis of HCC within 18 months (n=24); and (Panel C) Non-progressors and progressors with a diagnosis of HCC after 18 months of follow-up (n=14). Data are shown as mean+SEM of percentage. SFAs, saturated fatty acids; n3-PUFAs, omega-3 polyunsaturated fatty acids; n6-PUFAs, omega-6 polyunsaturated fatty acids.

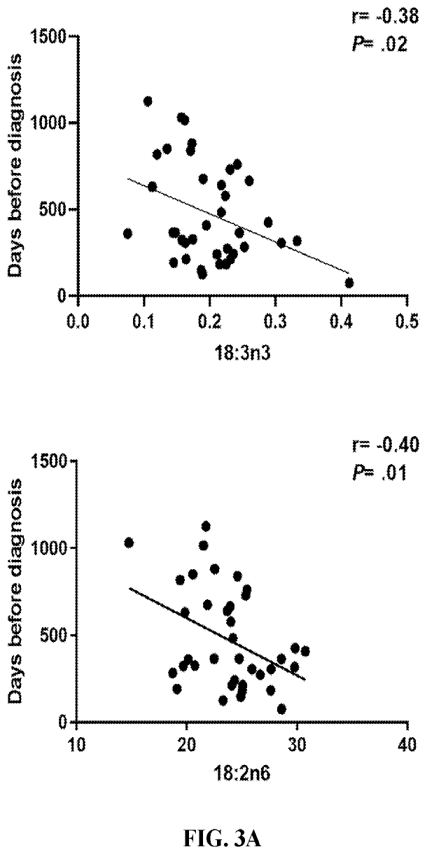

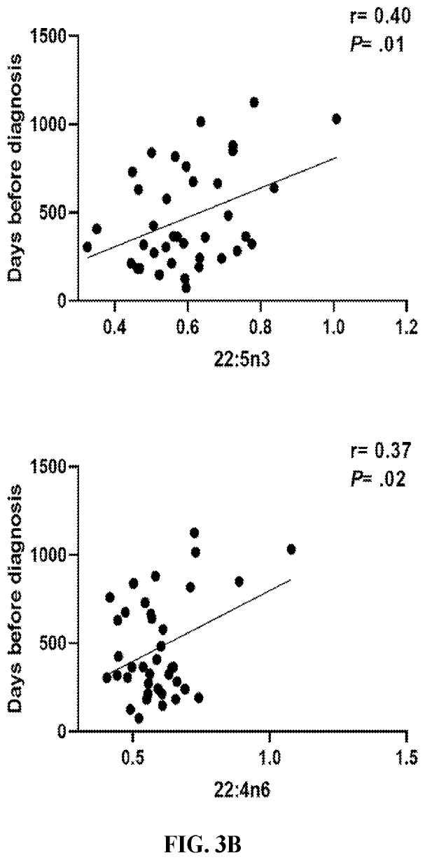

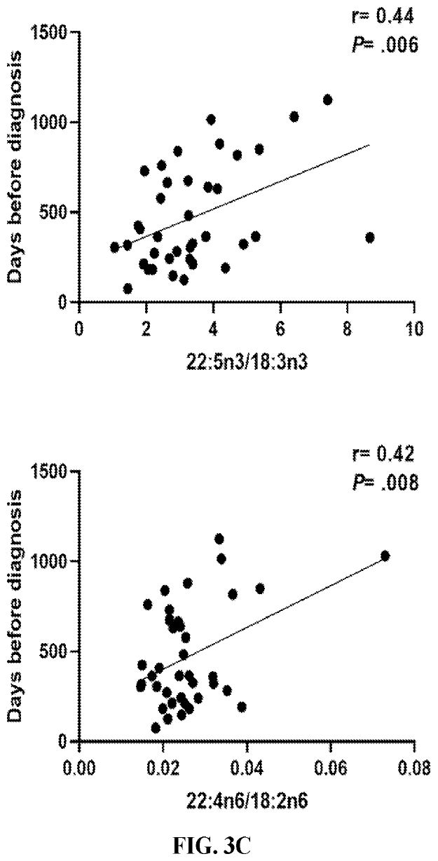

FIG. 3 shows correlations between selected FA levels and the time between baseline sample collection and HCC diagnosis in 40 patients with cirrhosis and HCC diagnosis during surveillance according to aspects of this disclosure. (Panel A) correlation between 18:3n3 (top), 18:2n6 (bottom) and days to HCC diagnosis; (Panel B) correlation between 22:5n3 (top), 22:4n6 (bottom) and days to HCC diagnosis; (Panel C) correlation between the ratio 22:5n3/18:3n3 (top), the ratio 22:4n6/18:2n6 (bottom) and days to HCC diagnosis. r, Pearson's correlation coefficient.

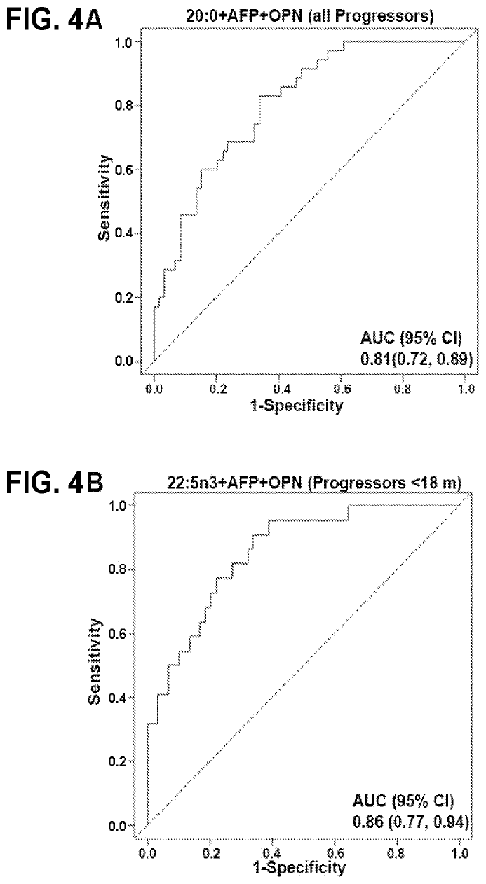

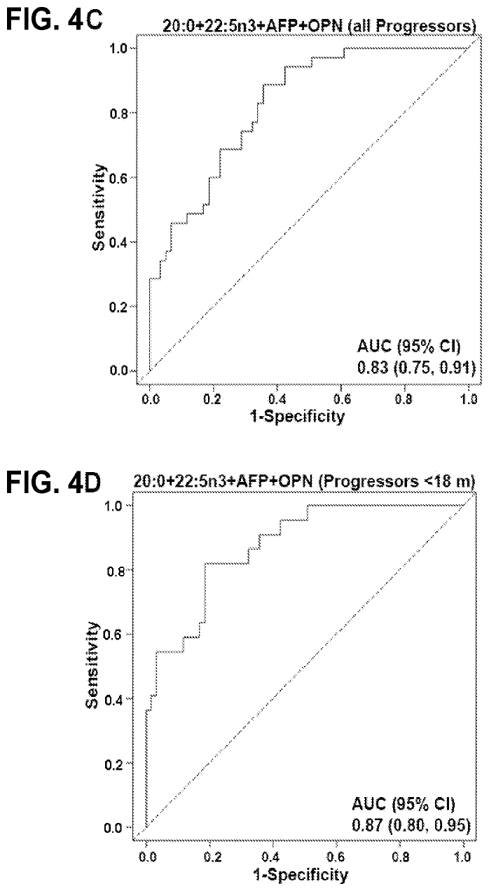

FIG. 4 shows ROC curves for biomarker combinations in non-progressors vs progressors according to aspects of this disclosure. (Panel A) ROC curve for combination of 20:0+AFP+OPN when all progressors were selected, (Panel B) ROC curve for combination of 22:5n3+AFP+OPN, when only progressors with a diagnosis of HCC within 18 months of surveillance were selected. (Panels C-D) ROC curves for combination of 20:0+22:5n3+AFP+OPN when all progressors (Panel C) and only progressors within 18 months to HCC diagnosis (Panel D) were selected. AFP, Alpha-fetoprotein; OPN, osteopontin; ROC, receiver operating characteristic; AUC, area under the curve; CI, confidence interval. P-values for AFP+OPN+20:0+22:5n3 vs AFP+OPN=0.05; AFP+OPN+20:0+22:5n3 vs AFP+OPN (<18 m)=0.05; AFP+OPN+20:0:+22:5n3 vs AFP=0.006; and AFP+OPN+20:0+22:5n3 vs AFP (<18 m)=0.003. For AFP+OPN+20:0+22:5n3, the coefficients are 0.012, 0.013, −10.125 and −3.569 for all progressors and 0.018, 0.015, −8.663 and −6.071 for progressors <18 m.

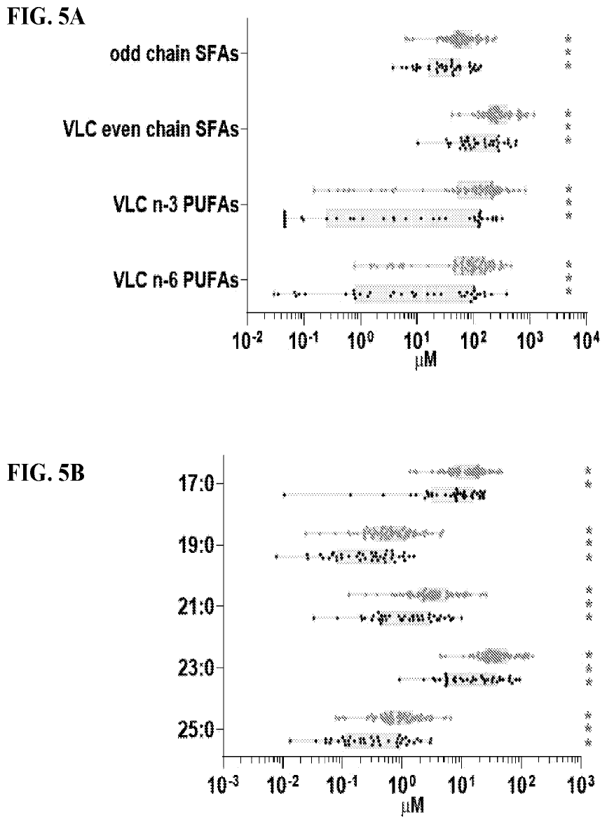

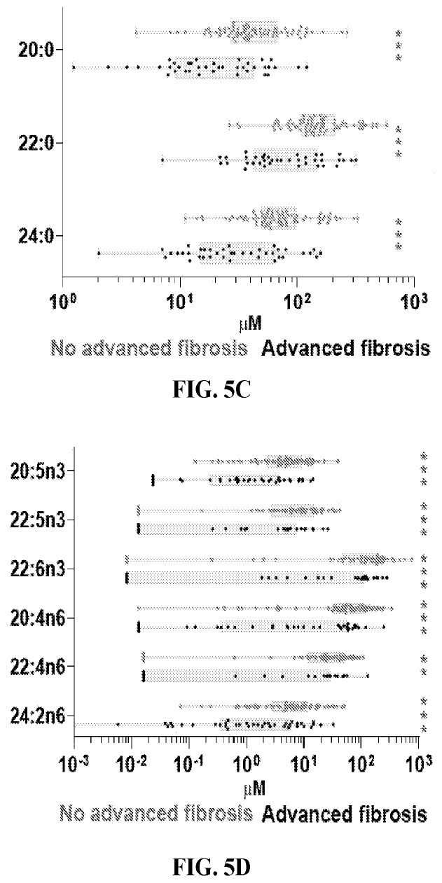

FIG. 5 shows subjects with advanced liver fibrosis had lower levels of odd chain SFAs, VLC even chain SFAs, VLC n-3 PUFAs and VLC n-6 PUFAs according to aspects of this disclosure. (Panel A) Concentrations of selected fatty acid groups. (Panel B) Individual odd chain SFAs. (Panel C) Individual VLC even chain SFAs. (Panel D) Individual VLC n-3 PUFAs and VLC n-6 PUFAs. **p≤0.01, ***p≤0.001. Boxes: range between first and third quartiles; Lines: median values; Whiskers: minimum and maximum values

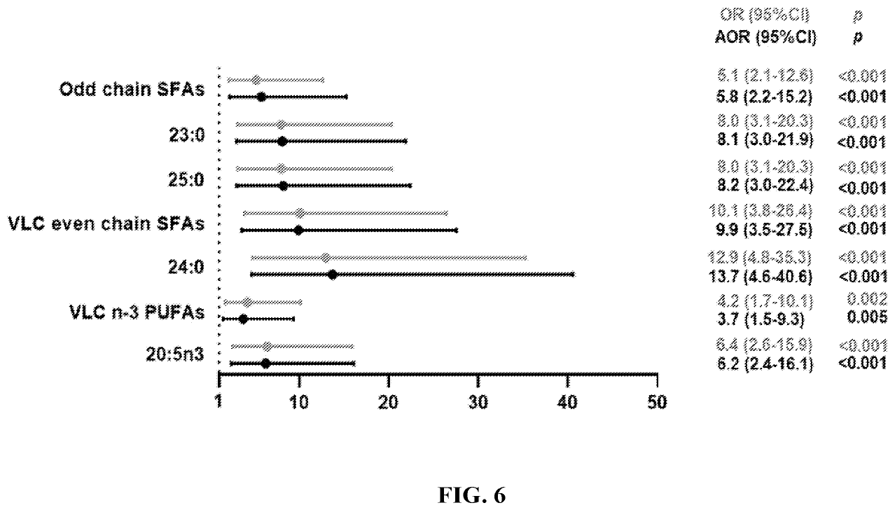

FIG. 6 shows forest plot of associations between low plasma concentrations (quartile Q1) of selected FFAs and advanced liver fibrosis according to aspects of this disclosure. AOR: adjusted for BMI, age and gender.

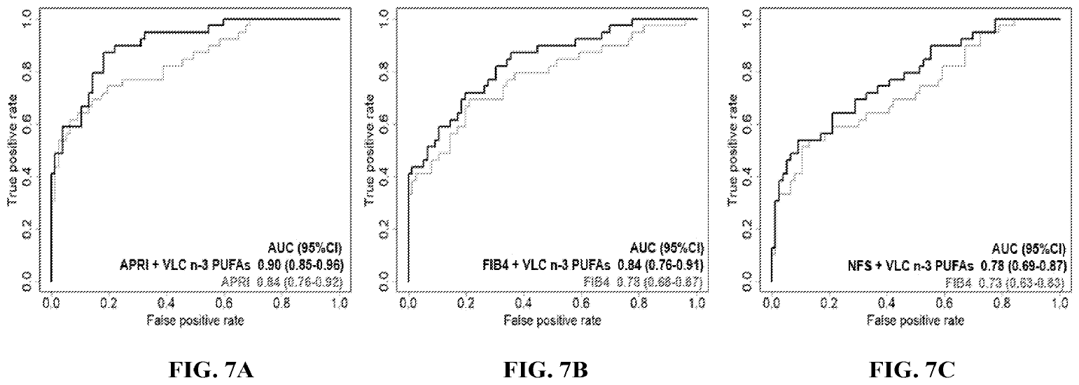

FIG. 7 shows ROC curves for the diagnosis of advanced liver fibrosis in CCHC subjects according to aspects of this disclosure. (Panel A) APRI alone and in combination with VLC n-3 PUFAs; (Panel B) FIB-4 alone and in combination with VLC n-3 PUFAs; (Panel C) NFS alone and in VLC PUFAs n3.

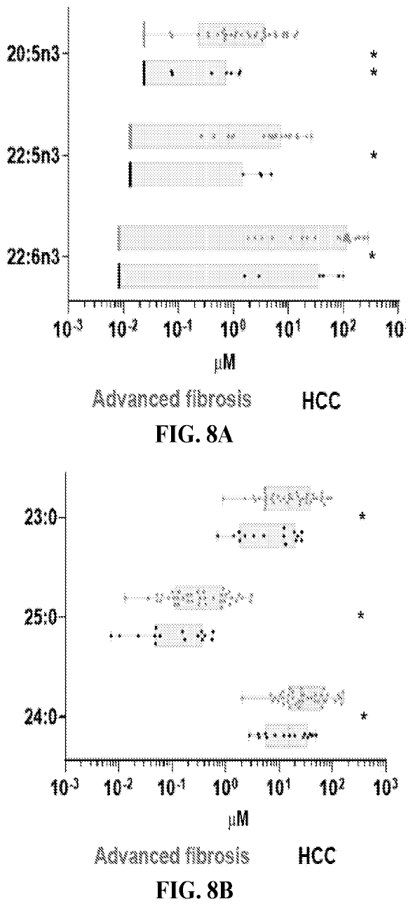

FIG. 8 shows HCC subjects with advanced fibrosis had lower levels of VLC n-3 PUFAs and VLC according to aspects of this disclosure. Concentrations of individual VLC n-3 PUFAs (Panel A) and of individual VLC SFAs (Panel B). *p≤0.05, **p≤0.01. Boxes: range between first and third quartiles; Lines: median values; Whiskers: minimum and maximum values.

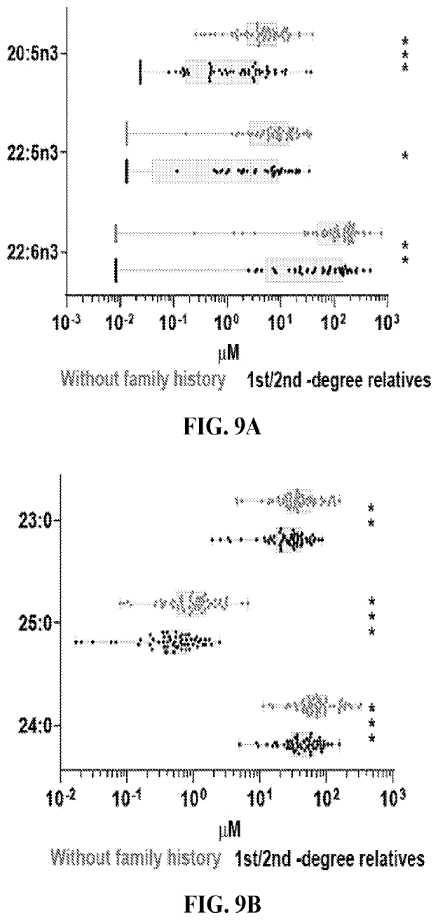

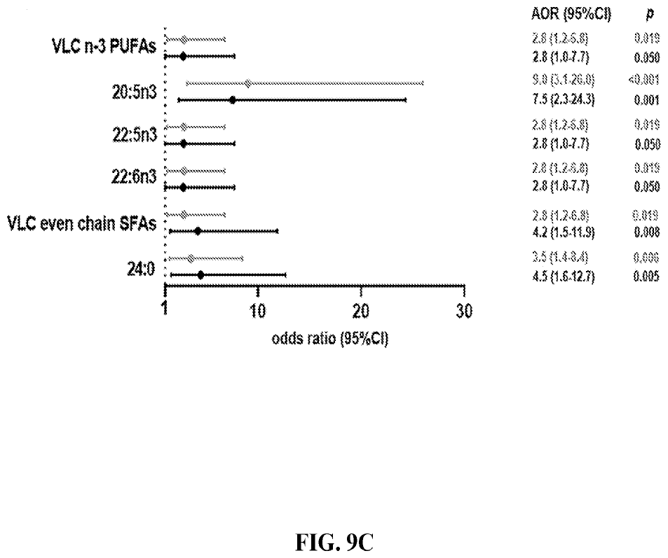

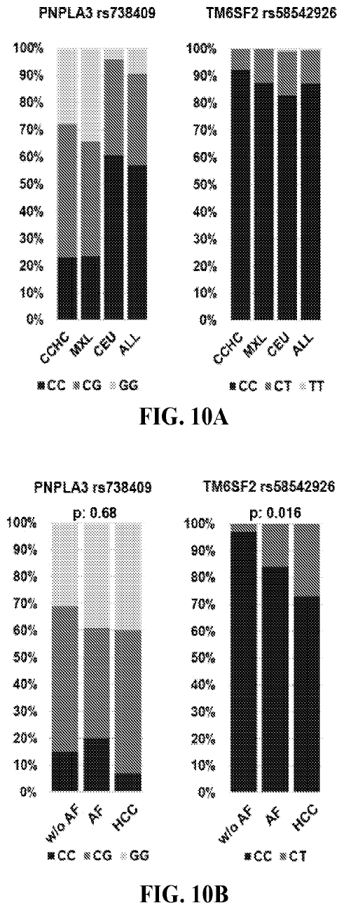

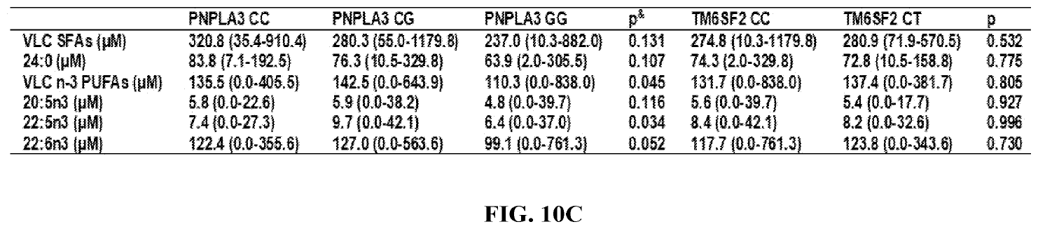

FIG. 9 shows non-fibrotic 1st/2nd-degree relatives of HCC patients had lower levels of VLC n-3 PUFAs and VLC SFAs according to aspects of this disclosure. Concentrations of individual VLC n-3 PUFAs (Panel A) and of individual VLC SFAs (Panel B). (Panel C) Forest plot of significant associations between being 1 st/2nd-degree relatives of HCC patients and selected low FFAs abundance (quartile Q1) among non-fibrotic subjects. Boxes: range between first and third quartiles; see FIG. 10. Association of PNPLA3 rs738409 and TM6SF2 rs58542926 with FFAs levels and advanced fibrosis or HCC. (Panel A) Genotype distribution of PNPLA3 rs738409 and TM6SF2 rs58542926 in CCHC and other populations. (Panel B) Genotype distribution of PNPLA3 rs738409 and TM6SF2 rs58542926 among different disease groups in study participants. (Panel C) Levels of selected FFAs in PNPLA3 rs738409 and TM6SF2 rs58542926 genotypes in CCHC subjects. MXL, Mexican Ancestry from Los Angeles, CEU, Utah Residents with Northern and Western European Ancestry; ALL, all individuals from 1000 Genomes Project. AF: advanced fibrosis; w/o AF: without advanced fibrosis. Data are presented as mean (range). &P comparing PNPLA3 GG versus PNPLA3 CC+CG. Lines: median values; Whiskers: minimum and maximum values. AOR: adjusted for age, gender and diabetic. *p≤0.05, **p≤0.01,***p≤0.001

FIG. 10 shows association of PNPLA3 rs738409 and TM6SF2 rs58542926 with FFAs levels and advanced fibrosis or HCC according to aspects of this disclosure. (Panel A) Genotype distribution of PNPLA3 rs738409 and TM6SF2 rs58542926 in CCHC and other populations. (Panel B) Genotype distribution of PNPLA3 rs738409 and TM6SF2 rs58542926 among different disease groups in study participants. (Panel C) Levels of selected FFAs in PNPLA3 rs738409 and TM6SF2 rs58542926 genotypes in CCHC subjects. MXL, Mexican Ancestry from Los Angeles, CEU, Utah Residents with Northern and Western European Ancestry; ALL, all individuals from 1000 Genomes Project. AF: advanced fibrosis; w/o AF: without advanced fibrosis. Data are presented as mean (range). &P comparing PNPLA3 GG versus PNPLA3 CC+CGgio.

### DETAILED DESCRIPTION

The present disclosure provides methods and compositions for detecting hepatocellular carcinoma (HCC) in biological samples from subjects. Provided herein is a method of detecting hepatocellular carcinoma (HCC) biomarkers in a sample by detecting in the sample one or more (e.g., two or more) hepatocellular carcinoma biomarkers. The present methods and compositions involve biomarkers identified from the analysis of biological samples. In particular embodiments, the methods are useful to detect HCC in subjects with liver cirrhosis or otherwise at risk of developing HCC. As such, the methods allow the detection of HCC in such subjects at the earliest possible stage, permitting more effective treatment. The methods are also useful for the management of patients with chronic liver disease (e.g., patient with non-alcoholic fatty liver diseases, NAFLD) to non-invasively stratify risk and identify patients with rapid disease progression.

The markers comprise different fatty acids, which can be used alone or in any combination to detect HCC in a subject. The HCC biomarkers are select fatty acids including the following: Tetracosanoic Acid, Heptadecanoic Acid, Eicosapentaenoic Acid, and Docosapentaenoic Acid. Additional or alternative HCC biomarkers to be detected in accordance with the method include 22:6n3-Docosahexaenoic acid, 20:0-Eicosanoic acid, 23:0 Tricosanoic acid, and/or 25:0-Pentacosanoic acid. The quantity of HCC biomarkers in the sample can be quantified to determine whether a threshold of increase or decrease has been achieved that provides an indication of the progression or regression of disease.

I. Subjects and Samples

The present methods and compositions can be used to detect HCC in a subject, e.g., a subject with one or more symptoms of HCC or with liver cirrhosis. In various embodiments, the subject may be an adult of any age, a child, or an adolescent. The subject may be male or female. In particular embodiments, the subject is a human.

“Hepatocellular carcinoma” or “HCC” refers to the most common type of primary liver cancer. As used herein, HCC can refer to HCC of any stage, e.g., stage 0, stage A, stage B, stage C and stage D of the Barcelona Clinic Liver Cancer classification. HCC as used herein encompasses all types of HCC, including fibrolamellar, pseudoglandular, pleomorphic, and clear cell types. HCC as used herein can encompass any growth pattern, including single large tumor, multiple tumors, or poorly defined tumors with infiltrative growth. In some embodiments, the present methods are used to screen for or detect HCC, e.g., early stage HCC, in a subject with a risk factor for HCC, including, but not limited to, cirrhosis, fibrosis, viral hepatitis (e.g., hepatitis B or C), exposure or consumption of one or more toxins (e.g., alcohol, aflatoxin, excessive iron as in hemochromatosis, pyrrolizidine alkaloids), one or more metabolic conditions (e.g., obesity, diabetes, nonalcoholic steatohepatitis), or congenital conditions such as alpha 1-antitrypsin deficiency. In particular embodiments, the methods are used to screen for HCC in subjects with liver cirrhosis, particularly advanced cirrhosis.

The subject may have one or more symptoms of HCC. A non-limiting list of symptoms includes nausea, loss of appetite, weight loss, fatigue, weakness, jaundice, swelling in the abdomen and/or legs, bruising or bleeding, white chalky stools, fever, abdominal pain, and others. The symptoms can be mild, moderate, or severe. In some embodiments, the subject may be considered at risk for developing HCC, even in the absence of symptoms. For example, the subject may have one or more risk factors such as a history of hepatitis B or C, of excessive alcohol consumption, obesity, diabetes, anabolic steroid use, iron storage disease, elevated consumption of aflatoxin, liver cirrhosis, liver fibrosis, or others. In particular embodiments, the subject has liver cirrhosis. The cirrhosis can be at any stage, e.g., early, intermediate, or advanced cirrhosis. In particular embodiments, the subject has advanced liver cirrhosis, and the methods are used to detect the appearance of HCC as early as possible. Nevertheless, an indication of HCC using the present methods can indicate any stage of HCC. In particular embodiments, the HCC that is detected is early stage HCC.

In one embodiment, the subject is a patient previously diagnosed with liver cirrhosis. In another embodiment, the subject is a patient previously diagnosed with liver fibrosis. In another embodiment, the subject is a patient previously diagnosed with liver fibrosis. In other embodiments the patient is a healthy patient, a patient suspected of having a liver disorder, a patient previously diagnosed with a liver disorder.

To assess the HCC biomarker status of the patient, a biological sample is obtained from the subject, particularly a bodily fluid. In some embodiments, the biological sample is a blood sample. In particular embodiments, the blood sample is plasma. In other embodiments, the blood sample is serum or whole blood. The bodily fluid from which the sample is obtained is preferably plasma, but can also be blood, sera or any other bodily fluid that contains fatty acids. Additional bodily fluids include, but are not limited to: urine, seminal fluid, vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, breast milk, amniotic fluid, bile, gastric juice, lymph, mucus, pericardial fluid, peritoneal fluid, pleural fluid, pus, saliva, sebum, serous fluid, sputum, sweat, and tears. The sample can be obtained from the subject using conventional techniques known in the art.

In one embodiment, if the subject currently undergoing treatment for HCC, at least two samples can be obtained at different time points during the treatment and one of at least one of the different time points is a time point prior to start of that treatment. In this way, detection of the HCC biomarkers described herein before and during treatment can be useful in determining whether or not that particular treatment is successfully treating HCC or not. Quantitative analysis of the HCC biomarkers over time can be determined using the method at multiple time points before, during and after treatment.

II. Selection of Biomarkers

Fatty acids are carboxylic acids typically found in lipids in plant and animal tissue. These are generally named according to the number of carbon atoms in the chain, and the number of double bonds in the chain, for example, C18: (octadecaenoic acid) contains 18 carbon atoms with a single double bond in the chain. Saturated fatty acids contain no double bonds. Mono unsaturated fatty acids contain one double bond, poly unsaturated fatty acids contain two or more. Trans fatty acids are unsaturated fatty acids in which at least one double bond is in the trans position as opposed to the more typical cis position. Trans fatty acids are rare in nature. Most fatty acids in food are bound to glycerol molecule as triglycerides, but others are common: namely monoglycerides, diglycerides, phospholipids, and free fatty acids.

The presence of HCC in a subject is determined by detecting levels of HCC biomarkers, e.g., HCC biomarkers, in a biological sample. As used herein, a “biomarker” refers to a molecule whose level in a biological sample, e.g., a blood sample such as a plasma sample, is correlated with the presence or absence of hepatocellular carcinoma (i.e., their “HCC status”). In particular embodiments, the biomarkers are fatty acids. The levels of each of the biomarkers need not be correlated with the HCC status in all subjects; rather, a correlation will exist at the population level, such that the level is sufficiently correlated within the overall population of individuals with HCC that it can be combined with the levels of other biomarkers, in any of a number of ways, as described elsewhere herein, and used to determine the HCC status. The values used for the measured level of the individual biomarkers can be determined in any of a number of ways, including direct readouts from relevant instruments or assay systems, e.g., using means known to those of skill in the art. In some embodiments, the readout values of the biomarkers are compared to the readout value of a reference or control, a fatty acid or other molecule whose level does not vary according to HCC status and whose level is measured at the same time as the biomarkers.

The term “correlating” generally refers to determining a relationship between one random variable with another. In various embodiments, correlating a given biomarker level with the presence or absence of HCC comprises determining the presence, absence or amount of at least one biomarker in a subject with the same outcome. In specific embodiments, a set of biomarker levels, absences or presences is correlated to a particular outcome, using receiver operating characteristic (ROC) curves.

The select fatty acids useful in the present method as HCC biomarkers include one or more (e.g., two or more) very long chain (VLC) saturated fatty acids (SFAs) and very long chain (VLC) n-3 and n-6 polyunsaturated fatty acids (PUFAs). Applicant unexpectedly discovered that detection of decreased plasma levels of very long chain n-3 polyunsaturated fatty acids and very long chain saturated fatty acids as HCC biomarkers that are indicative of HCC in patients having advanced liver fibrosis. In some embodiments, the HCC biomarkers include one or more (e.g., two or more) of Tetracosanoic Acid (24:0) (also known as lignoceric acid), Heptadecanoic Acid (17:0) (also known as margaric acid), Eicosapentaenoic Acid (20:5n3) (also known as icosapentaenoic acid), or Docosapentaenoic Acid (22:5n3). Additional exemplary HCC biomarkers include Docosahexaenoic acid (22:6n3). Additional exemplary HCC biomarkers include any of arachidic acid (20:0) (also known as icosanoic acid and eicosanoic acid), behenic acid (22:0) (also known as docosanoic acid), tricosylic acid (23:0) (also known as tricosanoic acid), lignoceric acid (24:0) (also known as tetracosanoic acid), and pentacosanoic acid (25:0). In some embodiments, the HCC biomarkers include n-3 polyunsaturated fatty acids 20:5n3, 22:5n3, and 22:6n3. In some embodiments, the HCC biomarkers include 20:5n3. In some embodiments, the HCC biomarkers include very long chain saturated fatty acids 24:0, 23:0, and 25:0. In some embodiments, the HCC biomarkers include 24:0. In some embodiments, the HCC biomarkers include n-3 polyunsaturated fatty acids 20:5n3, 22:5n3, and 22:6n3 and very long chain saturated fatty acids 24:0, 23:0, and 25:0. In some embodiments, the HCC biomarkers include n-3 polyunsaturated fatty acid 20:5n3 and very long chain saturated fatty acid 24:0. In some embodiments, the HCC biomarkers include arachidic acid (20:0), behenic acid (22:0), tricosylic acid (23:0), and lignoceric acid (24:0). In some embodiments, the HCC biomarkers include α-linoleic acid (18:3n3), eicosapentanenoic acid, docosapentaenoic acid, and/or docosahexanenoic acid. In some embodiments, the HCC biomarkers include α-linoleic acid (18:3n3) and eicosapentanenoic acid, docosapentaenoic acid, and/or docosahexanenoic acid. In some embodiments, the HCC biomarkers include at least two of saturated fatty acids 20:0, 22:0, 23:0, and/or 24:0. Any combination of the VLC saturated fatty acids and VLC n-3 and n-6 polyunsaturated fatty acids recited above can be used in the methods, kits, and systems provided herein.

The two or more hepatocellular carcinoma biomarkers detected by the method can further include osteopontin (OPN) and/or alpha-fetoprotein (AFP), especially when the patient from whom the sample is collected was previously diagnosed with liver fibrosis.

Any number of biomarkers can be assessed in the methods, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100 or more biomarkers.

In some embodiments, AUC values are used as a measure of the ability of a biomarker or combination of biomarkers to determine the HCC status of an individual. The “area under curve” or “AUC” refers to area under a ROC curve. AUC under a ROC curve is a measure of accuracy. An area of 1 represents a perfect test, whereas an area of 0.5 represents an insignificant test. For suitable biomarkers as described herein, the AUC may be between 0.700 and 1. For example, the AUC may be at least about 0.700, at least about 0.750, at least about 0.800, at least about 0.810, at least about 0.820, at least about 0.830, at least about 0.840, at least about 0.850, at least about 0.860, at least about 0.870, at least about 0.880, at least about 0.890, at least about 0.900, at least about 0.910, at least about 0.920, at least about 0.930, at least about 0.940, at least about 0.950, at least about 0.960, at least about 0.970, at least about 0.980, at least about 0.990, or at least about 0.995. In some embodiments, the selected biomarkers or combinations of biomarkers have an AUC score of at least about 0.6, 0.65, 0.7, 0.75, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or more with respect to their ability to distinguish between samples from patients with HCC vs non-HCC samples.

The biomarkers used in the present methods correspond to molecules whose levels within biological samples, e.g., blood samples, particularly plasma samples, from the subject correlate with the presence of hepatocellular carcinoma (HCC). The level of the individual biomarkers can be elevated or depressed in individuals with HCC relative to the level in individuals without HCC. What is important is that the level of the biomarker is positively or inversely correlated with HCC, allowing the determination of a diagnosis in a subject based on a measurement of the biomarker level in a sample from the subject as described herein.

HCC biomarkers can be assessed and identified using any standard analysis method or metric, e.g., by analyzing data from samples taken from subjects with or without a diagnosis of HCC, as described in more detail elsewhere herein and as illustrated, e.g., in the Examples. For example, in some embodiments, differences in AUC data between groups (e.g., between samples from HCC patients and samples from healthy patients without HCC) are evaluated using the Mann-Whitney U test. In some embodiments, principal component analysis (PCA) was performed with, e.g., the Euclidian-based distances matrix. Receiver operating characteristic (ROC) curves are generated, e.g., using the pROC package in R, and the AUCs calculated with a 95% confidence interval as well as sensitivity and specificity values. Binomial logistic regression analysis can be performed, e.g., for the analysis of combinations of multiple variables.

III. Detection

The levels of the biomarkers in the sample can be assessed in any of a number of ways including, but not limited to, gas chromatography, mass spectroscopy, gas chromatography-mass spectrometry, or liquid chromatograph-mass spectrometry. In some embodiments, free fatty acids present in the sample can be detected by gas chromatography (GC) or liquid chromatography (LC) and/or mass spectrometry (MS or mass spec). In particular embodiments, the levels of the biomarkers are determined using ultra-high resolution mass spectrometry. In some embodiments, an internal control is used, e.g., a reference molecule, e.g., lipid, whose level is known to not vary in correlation with the presence or absence of HCC. In some embodiments, one or more known biomarkers for HCC is assessed together with the herein-described biomarkers, e.g., alpha-fetoprotein or osteopontin.

Fatty acids are commonly analyzed by gas chromatography (GC) after conversion to fatty acid methyl esters (FAMEs), which are more easily separated and quantified than either triglycerides or free fatty acids. In most methods, the fat is saponified, thereby liberating the fatty acids from triglycerides, phospholipids, etc., producing free fatty acids. The free fatty acids are then trans-esterified to form fatty acid methyl esters. Matrices that are not pure fats and oils require an extraction step to liberate the fat for analysis. Most solid samples are hydrolyzed by strong acid and/or alkali, then extracted with organic solvents. In order to accurately quantify the fatty acid content of a sample as a weight percentage of sample, a synthetic fatty acid (typically C13:0, C19:0, C21:0 or C23:0) is added to the sample prior to extraction as an internal standard. The use of the internal standard compensates for variability in both the preparation and analysis of the sample. The fatty acid methyl esters are then separated using chromatographic techniques such as gas chromatography (GC) or liquid chromatography (LC) and quantified using a flame ionization detector (FID) or mass spectrometer (MS). Separations are performed with wax type capillary columns when only basic chain length and saturation are needed. In order to quantify cis versus trans isomerization, specialized, highly-polar capillary columns are used. If FID is utilized, the FID burns the FAMEs, producing ions that generate an electrical current that is measured and plotted as the response in the chromatogram.

In one embodiment, samples are prepared for detection by the following procedures or equivalents thereof: extracting free fatty acids from the sample, converting the free fatty acids to acyl chloride intermediates, derivatizing the acyl chloride intermediates to form derivatized products, and analyzing the derivatized products gas chromatography, by gas chromatography-mass spec (GC-MS) or by liquid chromatography-mass spectrometry (LC-MS) to detect the two or more fatty acid HCC biomarkers.

In one embodiment, the process of fatty acid extraction comprises: Use of an internal standard mixture consisting of 12.5 μg/mL of (1, 2, 3, 4, 5, 6-13C6) docosanoic acid and 25 μg/mL 13C-labeled myristic, palmitoleic, palmitic, margaric, linoleic, oleic, elaidic, and stearic acid in ethanol (Cambridge Isotope Laboratories, Tewksbury, MA, USA). To vials containing 20 μL of human plasma, 32 pL of internal standard mixture is added. The extraction solvent, methanol pre-cooled to −80° C. is then added (1 mL), and the solutions vortexed for 5 min. The tubes are allowed to sit on ice for 10 min, followed by centrifugation at 4,122 g, at 4° C., for 10 min. The supernatants are then transferred to 2 mL vials with Teflon caps and dried using a centrifugal vacuum concentrator.

In one embodiment, the process of fatty acid derivatization comprises: Conversion of extracted free fatty acid acids to acyl chloride intermediates by treatment with 200 μL of two molar oxalyl chloride in dichloromethane at 65° C. for five min. The solutions are then dried using a centrifugal vacuum concentrator. Dried samples are derivatized by adding 150 μL of 1% (v/v) 3-picolylamine in acetonitrile. The reaction is incubated at room temperature for 5 min. Finally, the solutions are dried using a centrifugal vacuum concentrator and stored at −80° C.

In some embodiments, the derivatized fatty acids are assessed using reverse phase liquid chromatography. In one embodiment, the processing method comprises: Reconstituting dried derivatization products in 100 μL ethanol, transferring them entirely to auto-sampler vials, drying them using a centrifugal vacuum concentrator, and then reconstituting them in 15 μL ethanol. An injection volume of 5 μL can be used. Mobile phase A (MPA) can be water containing 0.1% formic acid, and mobile phase B (MPB) can be acetonitrile containing 0.1% formic acid. The chromatographic method and system can include a Thermo Fisher Scientific Accucore C30 column (2.6 μm, 150×2.1 mm) maintained at 15° C., an autosampler tray chilling at 8° C., a mobile phase flowrate of 0.500 mL/min, and a gradient elution program as follows: 0-5 min, 65% MPB; 5-5.1 min, 65-90% MPB; 5.1-55 min, 90% MPB; 55-55.1, 90-65% MPB; 55.1-60 min, 65% MPB.

In some embodiments, the fatty acids are assessed using tandem mass spectrometry. In one embodiment, the method and system can include a Thermo Fisher Scientific Orbitrap Fusion Tribrid mass spectrometer with heated electrospray ionization source operated in data dependent acquisition mode with a scan range of 150-550 m/z, resolution of 240,000 (FWHM), positive ionization mode, a spray voltage of 3,500 V, and vaporizer and capillary temperatures set at 250° C. and 375° C., respectively. The sheath, auxiliary and sweep gas pressures can be 35, 10, and 0 (arbitrary units), respectively. Ions can be fragmented using the HCD cell with stepped collision energies of 27, 30, and 33%.

In some embodiments, the detection is carried out in whole or in part using an integrated system, as described elsewhere herein, which may also comprise a computer system as described elsewhere herein.

In some embodiments, replicates (e.g., triplicates) of any of the herein-described assays may be run for each sample in order to gain a higher level of confidence in the data. Replicate values can be averaged, and standard deviations can be calculated.

In some embodiments, the herein-described methods for detecting biomarker levels are performed multiple times for an individual subject. For example, in some embodiments, the subject is undergoing treatment for HCC (e.g., a drug treatment, radiation treatment, and/or surgical treatment), and the samples are obtained at different time points during the treatment to assess the efficacy of the treatment. In some embodiments, the subject is known to be or believed to be at risk for HCC, and the samples are obtained at different time points to detect a potential evolution in the risk for HCC and/or to detect HCC as early as possible.

In one embodiment, the detecting includes determining the concentrations of each of the one or more (e.g., two or more) biomarkers in the sample. Concentration is ideally obtained by comparing the results to a standard.

IV. Determining HCC Status

To determine the presence of HCC (i.e., the “HCC status”) in an individual (i.e., a subject or patient), the measured biomarker levels in a sample obtained from the individual are generally compared to reference levels, e.g., levels taken from a healthy individual without HCC. The reference control levels can be measured at the same time as the biomarker levels, i.e., using the same sample, or can be a level determined based on previous measurements.

Thus, in one aspect, provided herein is a method of diagnosing HCC in a subject comprising, consisting essentially of, or consisting of: detecting in a sample from the subject one or more (e.g., two or more) of any of the hepatocellular carcinoma biomarkers identified in Section II of this disclosure, wherein the sample is a sample of a bodily fluid obtained from a human subject. In some embodiments, the hepatocellular carcinoma biomarkers comprise one or more (e.g., two or more) of Tetracosanoic Acid, Heptadecanoic Acid, Eicosapentaenoic Acid, or Docosapentaenoic Acid.

In some embodiments, in a subject with advanced liver fibrosis, significantly lower levels of the following n-3 polyunsaturated fatty acids: 20:5n3, 22:5n3 and 22:6n3 and significantly lower levels of the following very long chain saturated fatty acids: 24:0, 23:0 and 25:0 indicate that the subject has HCC. The strongest associations are for 20:5n3 and 24:0. Supplements of very long chain n-3 polyunsaturated fatty acids or very long chain saturated fatty acids is indicated for prevention or treatment of cirrhosis and HCC.

In some embodiments, in a subject with cirrhosis, decreased plasma levels of HCC biomarkers arachidic acid (20:0), behenic acid (22:0), tricosylic acid (23:0), and lignoceric acid (24:0) indicate that the subject has HCC. In particular, higher levels of α-linoleic acid (18:3n3) and lower levels of eicosapentanenoic acid, docosapentaenoic acid, and/or docosahexanenoic acid indicate progression from cirrhosis to HCC. In some embodiments, increased levels of α-linoleic acid (18:2n6) and lower levels of arachidonic acid (20:4n6) and/or docosatetraenoic acid (22:4n6) indicate progression from cirrhosis to HCC in the subject. In some embodiments, detection of lower levels of at least two of the following four saturated fatty acids 20:0, 22:0, 23:0 and 24:0 indicates HCC progression from cirrhosis in the subject. In some embodiments, a combination of the detection of 20:0, 22:5n3, AFP and OPN further increased the ability to detect HCC in a sample.

When using multiple biomarkers, it is not necessary that all of the biomarkers are elevated or depressed relative to control levels in a sample, e.g., a plasma sample, from a given subject to give rise to a determination of HCC. For example, for a given biomarker level there can be some overlap between individuals falling into different probability categories. However, collectively the combined levels for all of the biomarkers included in the assay gives rise to an AUC score that indicates a high probability of, e.g., the presence of HCC.

In some embodiments, the levels of the selected biomarkers are quantified and compared to one or more preselected or threshold levels. Threshold values can be selected that provide an ability to predict the presence or absence of HCC. Such threshold values can be established, e.g., by calculating receiver operating characteristic (ROC) curves using a first population with HCC and a second population without HCC.

The present disclosure provides methods of generating a classifier(s) (also referred to as training) for use in the methods of determining the presence or absence of HCC in a subject. As used herein, the terms “classifier” and “predictor” are used interchangeably and refer to a mathematical function that uses the values of the signature (e.g. fatty acid levels from a defined set of biomarkers) and a pre-determined coefficient for each signature component to generate scores for a given observation or individual patient for the purpose of assignment to a category. A classifier is linear if scores are a function of summed signature values weighted by a set of coefficients. Furthermore, a classifier is probabilistic if the function of signature values generates a probability, a value between 0 and 1.0 (or 0 and 100%) quantifying the likelihood that a subject or observation belongs to a particular category or will have a particular outcome, respectively. Probit regression and logistic regression are examples of probabilistic linear classifiers.

A classifier, including a linear classifier, may be obtained by a procedure known as training, which consists of using a set of data containing observations with known category membership (e.g., subjects with HCC or without HCC). Specifically, training seeks to find the optimal coefficient for each component of a given signature, where the optimal result is determined by the highest classification accuracy. In some embodiments, a unique classifier may be developed and trained with respect to a particular platform upon which the signature is measured.

For example, classifiers that use fatty acid biomarker levels can be generated from a training set of samples obtained from patients having a known HCC status. Measurements of many fatty acid can be obtained, e.g., as disclosed elsewhere herein. The measurements can be analyzed to determine sets of biomarkers (i.e., their levels) that best discriminate between the different classifications of the training set via an optimization procedure. The analysis of fatty acid level data can include training a machine learning model to distinguish between positive and negative samples based on the levels of certain fatty acid biomarkers. The analysis can include using the data as a training set where the biomarker levels and known diagnosis are used to train a machine learning model to distinguish between positive and negative samples. In the process of learning, the model identifies fatty acid biomarkers that are predictive for HCC.

Hence, one aspect of the present disclosure provides a method of making an HCC classifier comprising, consisting of, or consisting essentially of (i) obtaining a biological sample such as a blood or plasma sample from a plurality of subjects suffering from HCC; (ii) measuring the levels of a plurality of fatty acid HCC biomarkers; (iii) normalizing the levels; (iv) generating a HCC classifier to include normalized biomarker levels and a “weighting” coefficient value; and optionally (v) uploading the classifier (e.g., fatty acid identity and weighing coefficient) to a database.

In some embodiments, the method further includes uploading the final fatty acid target list for the generated classifier, the associated weights (wₙ), and threshold values to one or more databases.

In some embodiments, the measuring comprises the detection and quantification (e.g., semi-quantification) of the selected biomarkers in the sample. The biomarker levels are typically normalized following detection and quantification as appropriate for the particular platform using methods routinely practiced by those of ordinary skill in the art. In some embodiments, the measured biomarker levels are adjusted relative to one or more standard level(s) (“normalized”). As known in the art, normalizing is done to remove technical variability inherent to a platform to give a quantity or relative quantity (e.g., of fatty acids).

In some embodiments, the measurement of differential levels of specific biomarkers from biological samples may be accomplished using a range of technologies, reagents, and methods. These include any of the methods of measurement as described elsewhere herein.

In some embodiments, the signatures may be obtained using a supervised statistical approach known as sparse linear classification in which sets of fatty acids are identified by the model according to their ability to separate phenotypes during a training process that uses the selected set of patient samples. The outcomes of training is a biomarker signature(s) and classification coefficients for the classification comparison. Together the signature(s) and coefficient(s) provide a classifier or predictor. Training may also be used to establish threshold or cut-off values.

Threshold or cut-off values can be adjusted to change test performance, e.g., test sensitivity and specificity. For example, the threshold for HCC may be intentionally lowered to increase the sensitivity of the test for HCC, if desired.

In some embodiments, the classifier generating comprises iteratively: (i) assigning a weight for each normalized biomarker level value, entering the weight and expression value for each biomarker into a classifier (e.g., a linear regression classifier) equation and determining a score for outcome for each of the plurality of subjects, then (ii) determining the accuracy of classification for each outcome across the plurality of subjects, and then (iii) adjusting the weight until accuracy of classification is optimized. Biomarkers having a non-zero weight are included in the respective classifier.

Determining the accuracy of classification may involve the use of accuracy measures such as sensitivity, specificity, positive predictive value (PPV), negative predictive value (NPV), accuracy, and area under the curve (AUC) of a Receiver Operating Characteristic (ROC) curve corresponding to the diagnostic accuracy of detecting or predicting HCC.

In some embodiments, the classifier is a linear regression classifier and said generating comprises converting a score of said classifier to a probability using a link function. As known in the art, the link function specifies the link between the target/output of the model (e.g., probability of HCC) and systematic components (in this instance, the combination of explanatory variables that comprise the predictor) of the linear model. It says how the expected value of the response relates to the linear predictor of explanatory variable.

In some embodiments, the classifiers that are developed during training and using a training set of samples are applied for prediction purposes to diagnose new individuals (“classification”). For each subject or patient, a biological sample is taken and the normalized biomarker levels (i.e., the relative amounts of biomarkers) in the sample of each of the biomarkers specified by the signatures found during training are the input for the classifier. In other embodiments, the classifier can also use the weighting coefficients discovered during training for each biomarker. As outputs, the classifiers are used to compute probability values. Each probability value may be used to determine the presence or absence of HCC in the subject.

In some embodiments, these values may be reported relative to a reference range that indicates the confidence with which the classification is made. In some embodiments, the output of the classifier may be compared to a threshold value, for example, to report a “positive” in the case that the classifier score or probability exceeds the threshold indicating the presence of HCC. If the classifier score or probability fails to reach the threshold, the result would be reported as “negative” for the respective condition.

It should be noted that a classifier obtained with one platform may not show optimal performance on another platform. This could be due to the promiscuity of probes or other technical issues particular to the platform. Accordingly, also described herein are methods to adapt a signature as taught herein from one platform for another.

It will be appreciated that for any particular biomarker, a distribution of biomarker levels for subjects with and without HCC may overlap. Under such conditions, a test does not absolutely distinguish the two populations (i.e., with or without HCC) with 100% accuracy, and the area of overlap indicates where the test cannot distinguish them. A threshold value is selected, above which the test is considered to be “positive” and below which the test is considered to be “negative.” The AUC of the ROC curve provides the C-statistic, which is a measure of the probability that the perceived measurement will allow correct identification of a condition (see, e.g., Hanley et al., Radiology 143: 29-36 (1982)).

In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more biomarkers are selected to discriminate between subjects with HCC and subjects without HCC with at least about 70%, 75%, 80%, 85%, 90%, 95% accuracy or having a C-statistic of at least about 0.70, 0.75, 0.80, 0.85, 0.90, or 0.95.

The phrases “assessing the likelihood” and “determining the likelihood,” as used herein, refer to methods by which the skilled artisan can predict the presence or absence of a condition (e.g., HCC) in a patient. The skilled artisan will understand that this phrase includes within its scope an increased probability that a condition (e.g., HCC) is present or absent in a patient; that is, that a condition is more likely to be present or absent in a subject. For example, the probability that an individual identified as having a specified condition actually has the condition can be expressed as a “positive predictive value” or “PPV.” Positive predictive value can be calculated as the number of true positives divided by the sum of the true positives and false positives. PPV is determined by the characteristics of the predictive methods of the present methods as well as the prevalence of the condition in the population analyzed. The statistical algorithms can be selected such that the positive predictive value in a population having a condition prevalence is in the range of 70% to 99% and can be, for example, at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that an individual identified as not having a specified condition or outcome actually does not have that condition can be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of the disease in the population analyzed. The statistical methods and models can be selected such that the negative predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a subject is determined to have a significant probability of having or not having a specified condition or outcome (e.g., HCC). By “significant probability” is meant that the subject has a reasonable probability (0.6, 0.7, 0.8, 0.9 or more) of having, or not having, a specified condition or outcome.

In some embodiments, a detection of HCC can be based not solely on biomarker levels, but can also take into account clinical and/or other data about the subject, e.g., clinical data about the subject's current medical state (e.g., the presence of cirrhosis or fibrosis, and the state of advancement of such cirrhosis or fibrosis), the presence of any symptoms characteristic of HCC, the medical history of the subject, the presence of one or more risk factors for HCC, and/or demographic data about the subject (age, sex, etc.).

V. HCC Treatment

The detection of HCC in a subject using the present methods can indicate the delivery of medical care appropriate for, e.g., the stage, form, or other properties of the detected HCC. In some embodiments, the subject receives treatment such as a drug treatment, radiation treatment, and/or surgical treatment. If the subject is currently undergoing treatment for HCC, after detection of the hepatocellular carcinoma biomarkers in a sample from the patient, the patient can be treated with or can continue to receive treatment with an anti-HCC treatment such as a drug treatment, a radiation treatment and/or a surgical treatment. More detail on each of these treatments is provided below.

Thus, in one aspect, provided herein is a method for treating HCC in a subject comprising, consisting essentially of, or consisting of: administering an effective amount of an anti-hepatocellular carcinoma treatment to a subject having differential levels of one or more hepatocellular carcinoma biomarkers in a sample from the subject (e.g., a blood sample) as compared to a control, wherein the one or more hepatocellular carcinoma biomarkers comprise one or more (e.g., two or more) of the fatty acid HCC biomarkers recited in Section II of this disclosure and, for example, listed in any one or more of Table 1, Table 2, Supplementary Table S2, or Supplementary Table S3.

In some embodiments, the method comprises: providing a sample (e.g., a blood sample) from the subject (e.g., a subject with advanced liver fibrosis); detecting the one or more (e.g., two or more) hepatocellular carcinoma biomarkers in the sample, and comparing the levels of the one or more hepatocellular carcinoma biomarkers to a control, wherein the levels of n-3 polyunsaturated fatty acid 20:5n3 and very long chain saturated fatty acid 24:0 are decreased relative to control levels determined from a sample (e.g., a blood sample) of a healthy individual without hepatocellular carcinoma.

In some embodiments, the method comprises: providing a sample (e.g., a blood sample) from the subject (e.g., a subject with advanced liver fibrosis); detecting the one or more (e.g., two or more) hepatocellular carcinoma biomarkers in the sample, and comparing the levels of the one or more hepatocellular carcinoma biomarkers to a control, wherein the levels of n-3 polyunsaturated fatty acids 20:5n3, 22:5n3 and 22:6n3 and very long chain saturated fatty acids 24:0, 23:0 and 25:0 are decreased, relative to control levels determined from a sample (e.g., a blood sample) of a healthy individual without hepatocellular carcinoma.

In some embodiments, the method comprises: providing a sample (e.g., a blood sample, particularly a plasma sample) from the subject (e.g., a subject with cirrhosis); detecting the one or more (e.g., two or more) hepatocellular carcinoma biomarkers in the sample, and comparing the levels of the one or more hepatocellular carcinoma biomarkers to a control, wherein the levels arachidic acid (20:0), behenic acid (22:0), tricosylic acid (23:0), and lignoceric acid (24:0), and/or the level of α-linoleic acid (18:3n3) and the levels of eicosapentanenoic acid, docosapentaenoic acid, and/or docosahexanenoic acid are elevated, and/or the levels of at least two of saturated fatty acids 20:0, 22:0, 23:0 and 24:0 are decreased, relative to control levels determined from a sample (e.g., a blood sample, particularly a plasma sample) of a healthy individual without hepatocellular carcinoma. In some embodiments, the method further comprises detection of AFP and OPN levels.

A. Surgical Treatment

The best option to cure HCC is with either surgical resection (removal of the tumor with surgery) or a liver transplant. If all cancer in the liver is completely removed, the patient will have the best outlook. Thus, in some embodiments, a patient with HCC as detected using the present methods receives surgical treatment for the HCC such as surgical resection or a liver transplant. Small liver cancers may also be treated with other types of treatment such as ablation or radiation.

In some embodiments, a patient with HCC as detected using the present methods receives partial hepatectomy. Partial hepatectomy is surgery to remove part of the liver. Only people with good liver function who are healthy enough for surgery and who have a single tumor that has not grown into blood vessels can have this operation. Imaging tests, such as CT or MRI with angiography are done first to see if the cancer can be removed completely. Still, sometimes during surgery the cancer is found to be too large or has spread too far to be removed, and the surgery that has been planned cannot be done.

Most patients with liver cancer in the United States also have cirrhosis. In someone with severe cirrhosis, removing even a small amount of liver tissue at the edges of a cancer might not leave enough liver behind to perform important functions. People with cirrhosis are typically eligible for surgery if there is only one tumor (that has not grown into blood vessels) and they will still have a reasonable amount (at least 30%) of liver function left once the tumor is removed. Doctors often assess this function by assigning a Child-Pugh score, which is a measure of cirrhosis based on certain lab tests and symptoms. Patients in Child-Pugh class A are most likely to have enough liver function to have surgery. Patients in class B are less likely to be able to have surgery. Surgery is not typically an option for patients in class C.

In some embodiments, a patient with HCC as detected using the present methods receives a liver transplant. When available, a liver transplant may be the best option for some people with liver cancer. Liver transplants can be an option for those with tumors that cannot be removed with surgery, either because of the location of the tumors or because the liver has too much disease for the patient to tolerate removing part of it. In general, a transplant is used to treat patients with small tumors (either 1 tumor smaller than 5 cm across or 2 to 3 tumors no larger than 3 cm) that have not grown into nearby blood vessels. It can also rarely be an option for patients with resectable cancers. With a transplant, not only is the risk of a second new liver cancer greatly reduced, but the new liver will function normally.

According to the Organ Procurement and Transplantation Network, about 1,000 liver transplants were done in people with liver cancer in the United States in 2016, the last year for which numbers are available. Unfortunately, the opportunities for liver transplants are limited. Only about 8,400 livers are available for transplant each year, and most of these are used for patients with diseases other than liver cancer. Increasing awareness about the importance of organ donation is an essential public health goal that could make this treatment available to more patients with liver cancer and other serious liver diseases.

Most livers used for transplants come from people who have just died. But some patients receive part of a liver from a living donor (such as a close relative) for transplant. The liver can regenerate some of its lost function over time if part of it is removed. Still, the surgery does carry some risks for the donor. About 370 living donor liver transplants are done in the United States each year. Only a small number of them are for patients with liver cancer.

People needing a transplant must wait until a liver is available, which can take too long for some people with liver cancer. In many cases a person may get other treatments, such as embolization or ablation, while waiting for a liver transplant. Or doctors may suggest surgery or other treatments first and then a transplant if the cancer reoccurs.

B. Ablation

In some embodiments, a patient with HCC as detected using the present methods is treated using ablation. Ablation is treatment that destroys liver tumors without removing them. These techniques can be used in patients with a few small tumors and when surgery is not a good option. They are less likely to cure the cancer than surgery, but they can still be very helpful for some people. These treatments are also sometimes used in patients waiting for a liver transplant. Ablation is best used for tumors no larger than 3 cm across. For slightly larger tumors (1 to 2 inches, or 3 to 5 cm across), it may be used along with embolization. Because ablation often destroys some of the normal tissue around the tumor, it might not be a good choice for treating tumors near major blood vessels, the diaphragm, or major bile ducts.

People getting this type of treatment typically do not need to stay in a hospital. Often, ablation can be done without surgery by inserting a needle or probe into the tumor through the skin. The needle or probe is guided into place with ultrasound or CT scan. Sometimes, though, to be sure the treatment is aimed at the right place, the ablation may be done in the operating room under general anesthesia and may need an incision similar to that made for a partial hepatectomy. Possible side effects after ablation therapy include abdominal pain, infection in the liver, fever and abnormal liver tests. Serious complications are uncommon, but they are possible.

In some embodiments, the ablation is radiofrequency ablation (RFA). Radiofrequency ablation is one of the most common ablation methods for small tumors. It uses high-energy radio waves. The doctor inserts a thin, needle-like probe into the tumor through the skin. A high-frequency current is then passed through the tip of the probe, which heats the tumor and destroys the cancer cells. In some embodiments, the ablation is microwave ablation (MWA). Microwave ablation uses the energy from electromagnetic waves to heat and destroy the tumor using a probe. In some embodiments, the ablation is cryoablation (cryotherapy). Cryoablation destroys a tumor by freezing it using a thin metal probe. The probe is guided into the tumor and then very cold gasses are passed through the probe to freeze the tumor which causes the cancer cells to die. In some embodiments, the ablation is ethanol (alcohol) ablation, e.g., percutaneous ethanol injection (PEI). This is also known as percutaneous ethanol injection (PEI). In this procedure, concentrated alcohol is injected directly into the tumor to damage cancer cells. Sometimes multiple treatments of alcohol ablation may be needed.

C. Embolization Therapy

In some embodiments, a patient with HCC as detected using the present methods is treated using embolization therapy. Embolization is a procedure that injects substances directly into an artery in the liver to block or reduce the blood flow to a tumor in the liver. The liver has two blood supplies. Most normal liver cells are fed by the portal vein, whereas a cancer in the liver is mainly fed by the hepatic artery. Blocking the part of the hepatic artery that feeds the tumor helps kill off the cancer cells, but it leaves most of the healthy liver cells unharmed because they get their blood supply from the portal vein. Embolization is an option for some patients with tumors that cannot be removed by surgery. It can be used for people with tumors that are too large to be treated with ablation (usually larger than 5 cm across) and who also have adequate liver function. It can also be used with ablation. Embolization can reduce some of the blood supply to the normal liver tissue, so it may not be a good option for some patients whose liver has been damaged by diseases such as hepatitis or cirrhosis.

In some embodiments, the embolization is trans-arterial embolization (TAE). During trans-arterial embolization a catheter is inserted into an artery in the inner thigh through a small cut and eased up into the hepatic artery in the liver. A dye is usually injected into the bloodstream to help the doctor watch the path of the catheter. Once the catheter is in place, small particles are injected into the artery to plug it up, blocking oxygen and key nutrients from the tumor.

In some embodiments, the embolization is trans-arterial chemoembolization (TACE). Trans-arterial chemoembolization is usually the first type of embolization used for large liver cancers that cannot be treated with surgery or ablation. It combines embolization with chemotherapy. Most often, this is done by giving chemotherapy through the catheter directly into the artery, then closing up the artery, so the chemotherapy can remain in proximity to the tumor.

In some embodiments, the embolization is drug-eluting bead chemoembolization (DEB-TACE). Drug-eluting bead chemoembolization combines TACE embolization with drug-eluting beads. The procedure is essentially the same as TACE except that the artery is blocked after drug-eluting beads are injected. Because the chemotherapy is physically close to the cancer and because the drug-eluting beads slowly release the chemo, the cancer cells are more likely to be damaged and die. The most common chemotherapy drugs used for TACE or DEB-TACE are mitomycin C, cisplatin, and doxorubicin.

In some embodiments, the embolization is radioembolization (RE). Radioembolization combines embolization with radiation therapy. This is done by injecting microspheres having a radioactive isotope (yttrium-90 or Y-90) attached into the hepatic artery. Once infused, the beads lodge in blood vessels near the tumor, where they release small amounts of radiation to the tumor site for several days. The radiation travels a very short distance, so its effects are limited mainly to the tumor.

D. Radiation Therapy

In some embodiments, a patient with HCC as detected using the present methods is treated using radiation therapy. Radiation therapy uses high-energy rays, or particles to destroy cancer cells. This option may not be advised for the patient whose liver has been greatly damaged by disease such as hepatitis or cirrhosis. Radiation can be helpful in treating: liver cancer that cannot be removed by surgery; liver cancer that cannot be treated with ablation or embolization or did not respond well to those treatments; liver cancer that has spread to other areas such as the brain or bones; patients experiencing severe pain due to large liver cancers; patients having a tumor thrombus blocking the portal vein.

E. Drug Therapy

In some embodiments, a patient with HCC as detected using the present methods is treated using drug therapy, e.g., targeted drug therapy, immunotherapy, or chemotherapy. Targeted drugs work differently from standard chemotherapy drugs and include the following: kinase inhibitors; Sorafenib (Nexavar), lenvatinib (Lenvima), Regorafenib (Stivarga), cabozantinib (Cabometyx), Immunotherapy can comprise the administration of monoclonal antibodies. Monoclonal antibodies are designed to attach to a specific target. The monoclonal antibodies used to treat liver cancer affect a tumor's ability to form new blood vessels, also known as angiogenesis. These therapeutics are often referred to angiogenesis inhibitors and include: Bevacizumab (Avastin), which can be used in conjunction with the immunotherapy drug atezolizumab (Tecentriq); and Ramucirumab (Cyramza).

An important part of the immune system is its ability to keep itself from attacking normal cells in the body. To do this, it uses “checkpoints”—proteins on immune cells that need to be switched on or off to start an immune response. Cancer cells sometimes use these checkpoints to avoid being attacked by the immune system. Newer drugs that target these checkpoints hold a lot of promise as liver cancer treatments and include: PD-1 and PD-L1 inhibitors; Atezolizumab (Tecentriq), which can be used in conjunction with the targeted drug bevacizumab (Avastin); Pembrolizumab (Keytruda) and nivolumab (Opdivo), alone or in combination with ipilimumab (described below) may also be an option. Ipilimumab (Yervoy® blocks CTLA-4, another protein on T cells that normally helps keep them in check. This drug can be used in combination with nivolumab to treat liver cancer that has previously been treated, such as with the targeted drug sorafenib (Nexavar®). The combination of the two drugs may help shrink the cancer more than nivolumab alone.

The most common chemotherapy drugs for treating liver cancer include: Gemcitabine (Gemzar); Oxaliplatin (Eloxatin); Cisplatin; Doxorubicin (pegylated liposomal doxorubicin); 5-fluorouracil (5-FU); Capecitabine (Xeloda); Mitoxantrone (Novantrone), or combinations thereof. Chemotherapy can be regional when drugs are inserted into an artery that leads to the part of the body with the tumor. thereby focusing the chemo on the cancer cells in that area and reducing side effects by limiting the amount of drug reaching the rest of the body. Hepatic artery infusion (HAI), or chemo given directly into the hepatic artery, is an example of a regional chemotherapy that can be used for liver cancer. It is slightly different from chemoembolization because surgery is needed to put an infusion pump under the skin of the abdomen. The pump is attached to a catheter that connects to the hepatic artery. This is done while the patient is under general anesthesia. The chemo is injected with a needle through the skin into the pump' reservoir and it is released slowly and steadily into the hepatic artery. The drugs most commonly used for HAI include floxuridine (FUDR), cisplatin, and oxaliplatin.

VI. Kits, Other Methods, and Systems

A. Kits

In one aspect, kits are provided for the detection of HCC in a subject, wherein the kits can be used to detect the biomarkers described herein. The kit may include, e.g., one or more agents for the detection of biomarkers, a container for holding a biological sample, e.g., plasma sample, isolated from a human subject suspected of having HCC; and instructions for reacting agents with the biological sample or a portion of the biological sample to detect the presence or amount of at least one biomarker in the biological sample. The agents may be packaged in separate containers. The kit may further comprise one or more control reference samples and reagents for performing the herein-described methods. The kit may also comprise one or more devices or implements for carrying out any of the herein methods.

In certain embodiments, the kit comprises agents for measuring the levels of one or more (e.g., two or more) of the HCC biomarkers described in Section II of this disclosure and, for example, listed in any one or more of Table 1, Table 2, Supplementary Table S2, or Supplementary Table S3.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing instructions for methods of diagnosing HCC.

B. Measurement Systems and Reports for Detecting and Recording Biomarker Expression

In one aspect, a system, e.g., measurement system is provided. Such systems allow, e.g., the detection of biomarker levels in a sample and the recording of the data resulting from the detection. The stored data can then be analyzed to determine the HCC status of a subject. Such systems can comprise, e.g., assay systems (e.g., comprising an assay device and detector), which can transmit data to a logic system (such as a computer or other system or device for capturing, transforming, analyzing, or otherwise processing data from the detector). The logic system can have any one or more of multiple functions, including controlling elements of the overall system such as the assay system, sending data or other information to a storage device or external memory, and/or issuing commands to a treatment device.

Also provided is a system for detecting hepatocellular carcinoma biomarkers in a sample, by utilizing a station for analyzing the sample by mass spectrometry (Mass Spec or MS) or liquid chromatography/mass spectrometry (LC/MS) to detect one or more (e.g., two or more) hepatocellular carcinoma biomarkers in the sample, wherein the one or more (e.g., two or more) fatty acid HCC biomarkers are Tetracosanoic Acid, Heptadecanoic Acid, Eicosapentaenoic Acid, and/or Docosapentaenoic Acid, and the sample is a sample of a bodily fluid obtained from a subject (e.g., a blood sample such as a plasma sample). Optionally, a station for generating a report containing information on results of the analyzing is further included.

Also provided is a method of generating a report containing information on results of the detection of hepatocellular carcinoma biomarkers in a sample, including detecting one or more (e.g., two or more) hepatocellular carcinoma biomarkers in the sample, and generating the report, wherein the one or more (e.g., two or more) fatty acid HCC biomarkers are Tetracosanoic Acid, Heptadecanoic Acid, Eicosapentaenoic Acid, and/or Docosapentaenoic Acid; the sample is a sample of a bodily fluid obtained from a subject (e.g., a blood sample such as a plasma sample), and the report is useful for diagnosing hepatocellular carcinoma in the subject.

C. Computer/Diagnostic Systems for Determining HCC Status

Certain aspects of the herein-described methods may be totally or partially performed with a computer system including one or more processors, which can be configured to perform the steps. Thus, embodiments are directed to computer systems configured to perform the steps of methods described herein, potentially with different components performing a respective step or a respective group of steps. The computer systems of the present disclosure can be part of a measuring system as described above, or can be independent of any measuring systems. In some embodiments, the present disclosure provides a computer system that uses inputted biomarker expression (and optionally other) data, and determines the HCC status of a subject.

A computer system can include desktop and laptop computers, tablets, mobile phones and other mobile devices. The system can include various elements such as a printer, keyboard, storage device(s), monitor (e.g., a display screen, such as an LED), peripherals, devices to connect a computer system to a wide area network such as the Internet, a mouse input device, scanner, a storage device(s), computer readable medium, camera, microphone, accelerometer, and the like. Any of the data mentioned herein can be output from one component to another component and can be output to the user.

In one aspect, the present disclosure provides a computer implemented method for determining the presence or absence of HCC in a patient. The computer performs steps comprising, e.g., receiving inputted patient data comprising values for the levels of one or more biomarkers in a biological sample from the patient; analyzing the levels of one or more biomarkers and optionally comparing them to respective reference values, optionally comparing the biomarker levels to one or more threshold values to determine HCC status; and displaying information regarding the HCC status or probability in the patient. In certain embodiments, the inputted patient data comprises values for the levels of a plurality of biomarkers in a biological sample from the patient, e.g., biomarkers comprising one or more pairs or three-way combinations of the HCC biomarkers described in Section II of this disclosure and, for example, listed in any one or more of Table 1, Table 2, Supplementary Table S2, or Supplementary Table S3, and/or for any combination comprising two or more of the HCC biomarkers described in Section II of this disclosure and, for example, listed in any one or more of Table 1, Table 2, Supplementary Table S2, or Supplementary Table S3.

In a further aspect, a diagnostic system is included for performing the computer implemented method, as described. A diagnostic system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining the HCC status of the subject. For example, the storage component includes instructions for determining HCC status based on biomarker levels, as described herein. The computer processor is coupled to the storage component and configured to execute the instructions stored in the storage component in order to receive patient data and analyze patient data according to one or more algorithms. The display component displays information regarding the diagnosis of the patient. The storage component may be of any type capable of storing information accessible by the processor, such as a hard-drive, memory card, ROM, RAM, DVD, CD-ROM, USB Flash drive, write-capable, and read-only memories.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data. In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel. In one aspect, computer is a server communicating with one or more client computers. Each client computer may be configured similarly to the server, with a processor, storage component and instructions. Although the client computers and may comprise a full-sized personal computer, many aspects of the system and method are particularly advantageous when used in connection with mobile devices capable of wirelessly exchanging data with a server over a network such as the Internet.

### Examples

The present invention may be better understood by reference to the following non-limiting examples. The following example will serve to further illustrate the present invention without, at the same time, however, constituting any limitation thereof. On the contrary, it is to be clearly understood that resort may be had to various embodiments, modifications and equivalents thereof which, after reading the description herein; may suggest themselves to those skilled in the art without departing from the spirit of the invention.

### Example 1—Detection of HCC Biomarker in Samples from Patients with Cirrhosis

The present experiment was designed to further validate the performance of OPN and identify fatty acids (FAs) that could improve OPN's performance in HCC risk assessment in patients with cirrhosis.

A. Materials and Methods

Human subjects. Serum samples were collected from patients with cirrhosis enrolled at the University of Michigan and at the Michael E. DeBakey Veterans Affairs Medical Center. At both sites, patients with Child-Pugh class A or B cirrhosis without detectable HCC were enrolled in surveillance program using ultrasound and AFP every six months. If a suspicious lesion was observed on ultrasonography or AFP >20 ng/ml was detected, triple-phase computed tomography or magnetic resonance imaging were performed to further evaluate the mass. HCC diagnosis was made using the American Association for the Study of Liver Disease guidelines (Heimbach, J. K., et al. AASLD guidelines for the treatment of hepatocellular carcinoma. Hepatology. 2018; 67(1):358-80). The research protocol was approved by respective institutional review boards at both sites. All study participants provided written informed consent. A total of 103 patients were selected for this study, including 40 patients who developed HCC during follow-up. The remaining 63 patients did not develop HCC during follow-up and were selected based on the average and frequency of age, gender, race and etiology of the progressors as well as time of enrollment.

Osteopontin quantification. Serum concentrations of OPN were measured using a commercial ELISA kit from R&D Systems (Minneapolis, MN) as previously described (Shang, S., et al. Identification of osteopontin as a novel marker for early hepatocellular carcinoma. Hepatology. 2012; 55(2):483-90). Briefly, 50 IA of diluted (1:100) serum samples were added to the ELISA plates pre-coated with a capture OPN antibody.

Fatty acids profiling. Total lipids were extracted from 100 μL of the baseline serum samples and separated using gas chromatography (Agilent 7890 Gas Chromatograph with flame ionization detector) detector and ChemStation software; Supelco fused silica 100 m capillary column SP-2560). FAs were measured in the phospholipid fraction and their composition was expressed as weight percentage of the total phospholipid FA analyzed. The assay generated data on 46 FAs as previously described (Muir, K., et al. Proteomic and lipidomic signatures of lipid metabolism in NASH-associated hepatocellular carcinoma. Cancer Research. 2013; 73(15):4722-31). All assays were performed blinded to clinical data.

Statistical analysis. The demographic and clinical characteristics of the two study groups were compared using two-tailed student's t-test for continuous variables and Chi squared tests for categorical variables. Logistic regression was performed to predict sample status (progressors vs. non-progressors) based on continuous biomarker levels. First, univariate logistic regression models were fitted and we expanded the model by adding additional biomarkers. For each logistic regression model, the area under the receiver operating characteristic (ROC) curve (AUC) was calculated. In order to compare models, a likelihood ratio test was conducted. A P value of <0.05 was considered to be statistically significant. Correlations between the biomarkers as well as between biomarkers and time of diagnosis were assessed using Pearson's correlation analysis. All results were given as two-tailed p-values, and p<0.05 was considered statistically significant.

B. Results

1. Patients Study Characteristics

A total of 103 patients with cirrhosis without detectable HCC and enrolled in surveillance programs were selected for this study. Baseline clinical characteristics of these 103 patients are described in Supplementary Table S1. During follow-up, 40 patients developed HCC (progressors). The remaining 63 cirrhosis patients (non-progressors) correspond to patients free of HCC during the same follow-up period and selected based on the average and frequency of age, gender, race and etiologies of the progressors as well as time of enrollment. Indeed, there was no significant differences in age, gender, race, and etiology of the disease between the two groups. The median age of the study participants at baseline was 56 years (range: 42-69) in progressors and 58 (range: 44-67) in non-progressors. The majority of the study participants were male (90% and 94% in progressors and non-progressors, respectively) and the most common etiology of cirrhosis was HCV (75% in progressors and 84% in non-progressors). The median time between enrollment and HCC diagnosis was 365 days (range: 75-1125 days).

AFP concentration was significantly higher (p=0.03) in the prediagnostic serum samples collected from progressors at baseline (median=8.20 ng/ml; range: 2-132 ng/ml) compared to baseline sera from non-progressors (median=4.39 ng/ml; range: 0-54 ng/ml) (Supplementary Table S1; data are presented as median (range) or as frequency (%)). Among the 40 patients who developed HCC, only 10 (25%) had AFP values greater than 20 ng/ml. Among progressors who developed HCC within 18 months of follow-up (n=25), 7 (28%) had AFP levels above 20 ng/ml. When only progressors who developed HCC more than 18 months after enrollment were included in the analysis, AFP levels were not significantly different between progressors and non-progressors (p=0.19), with 21% of progressors and 10% of non-progressors with AFP values greater than 20 ng/ml.

SUPPLEMENTARY TABLE S1
Baseline clinical characteristics of the 103 cirrhotic study participants

| | | |
|---|---|---|
| Non- | | |
| progressors | Progressors | p |
| (n = 63) | (n = 40) | value |

| | | | | | |
|---|---|---|---|---|---|
| Age | 58 | (44-67) | 56 | (42-69) | 0.09 |
| Gender (% Male) | 59 | (93.65%) | 36 | (90.00%) | 0.98 |
| Race | | | | | 0.64 |
| Caucasian | 37 | (58.73%) | 25 | (62.50%) | |
| Black | 22 | (34.92%) | 11 | (27.50%) | |
| Hispanic | 4 | (6.35%) | 4 | (10.00%) | |
| Etiology | | | | | 0.66 |
| HCV | 53 | (84.13%) | 30 | (75.00%) | |
| PBC | 3 | (4.76%) | 3 | (7.50%) | |
| Alcohol | 4 | (6.35%) | 3 | (7.50%) | |
| NASH | 3 | (4.76%) | 4 | (10.00%) | |
| MELD Score | 7.56 | (0.94-17.00) | 9.21 | (1.56-18.95) | 0.04 |
| Creatinine | 1.77 | (0,60-4.60) | 1.98 | (0.60-4.20) | 0.39 |
| INR | 1.83 | (0.91-4.60) | 2.09 | (0.93-4.20) | 0.27 |

| | | | |
|---|---|---|---|
| Days to HCC diagnosis | N/A | 365 | (75-1125) |

| | | | | | |
|---|---|---|---|---|---|
| AFP | 4.39 | (0-54) | 8.20 | (2-132) | 0.03 |

HCV, hepatitis C virus; PBC, primary biliary cirrhosis; MELD, model for end-stage liver disease; INR, the international normalized ratio; AFP, Alpha-fetoprotein.

2. OPN Performance in Detecting Preclinical HCC in Cirrhotic Patients Who Developed HCC During Follow Up

We measured OPN, an early HCC biomarker we have identified in prior studies. Circulating OPN levels were significantly higher in prediagnostic serum samples collected from progressors at baseline (median=86.53 ng/ml; range: 43-256 ng/ml) than in baseline sera from non-progressors (median=67.72 ng/ml; range: 14-245 ng/ml) (p=0.006) (FIG. 1). Among the 40 progressors, 17 (42.5%) had OPN values above 90 ng/ml, the optimum cut-off concentration for HCC detection we previously reported (Shang, S., et al., 2012 Hepatology, supra), compared to 21% of the non-progressors. Among progressors who developed HCC within 18 months of follow-up (n=25), 12 (48%) had OPN levels above 90 ng/ml. When only progressors who developed HCC more than 18 months after enrollment were included in the analysis, OPN concentrations were not significantly different between progressors and non-progressors (FIG. 1), confirming that as for AFP, circulating OPN levels increase in a subset of patients with cirrhosis closest to HCC development.

ROC analysis showed a greater performance of OPN in HCC risk prediction compared to AFP and a complementarity performance between AFP and OPN. The area under ROC curve (AUC) for AFP alone was 0.67 [95% CI: 0.55-0.78] (P=0.002) while AUC for OPN alone was 0.71 [95% CI: 0.61-0.82] (P=0.002) (Table 1). AUC was the highest when AFP and OPN were combined (AUC=0.73 [95% CI: 0.63-0.84]; P=0.0006) (Table 1). The performance of OPN to detect preclinical disease was higher when only progressors who developed HCC within 18 months of follow-up were included: AUCs of OPN alone and in combination with AFP increased to 0.76 [95% CI: 0.66-0.87] (P=0.002) and to 0.77 [95% CI: 0.66-0.88] (P=0.0008), respectively (Table 1).

TABLE 1
Performance characteristics for OPN and AFP.

| | Non-progressors | | Non-progressors | |
|---|---|---|---|---|
| | vs Progressors | | vs Progressors (<18 m) | |
| | AUC | p- | AUC | p- |
| Biomarkers | [95% CI] | value | (95% CI) | value |
| AFP | 0.67 | 0.002 | 0.68 | 0.004 |
| | [0.55-0.78] | | [0.55-0.81] | |
| OPN | 0.71 | 0.002 | 0.76 | 0.002 |
| | [0.61-0.82] | | [0.66-0.87] | |
| AFP + | 0.73 | 0.0006 | 0.77 | 0.0008 |
| OPN | [0.63-0.84] | | [0.66-0.88] | |

AFP, Alpha-fetoprotein; OPN, osteopontin; AUC, area under the curve; CI, confidence interval.
p [AFP + OPN vs AFP] = 0.20;
p [AFP + OPN vs AFP (<18 m)] = 0.10

3. Circulating Free FAs Levels in Preclinical HCC Progressors and Cirrhosis Non-Progressors

To investigate whether circulating free FAs could detect preclinical HCC in patients with cirrhosis, FA profiling was performed in baseline sera from the same progressors and non-progressors, blinded to clinical data. Circulating levels of four SFAs, arachidic (20:0), behenic (22:0), tricosylic (23:0) and lignoceric (24:0) acids, were significantly lower in progressors who developed HCC during follow-up compared to cirrhosis non-progressors (p<0.0001 for all 4 FAs) (FIG. 2A). While no difference was observed in MUFAs, significant changes were detected in n-3 and n-6 PUFAs. Progressors had higher levels of α-linoleic acid (18:3n3) (p=0.006) and lower levels of eicosapentaenoic acid (EPA; 20:5n3) (p<0.0001), docosapentaenoic acid (DPA-n3; 22:5n3) (p<0.0001) and docosahexaenoic acid (DHA; 22:6n3) (p=0.009) compared to non-progressors (FIG. 2A). Linoleic acid (18:2n6) was also increased (p<0.0001) in progressors compared to non-progressors, while levels of arachidonic (20:4n6) and docosatetraenoic (22:4n6) acids were lower (p=0.002 and p=0.04, respectively).

To evaluate the relation between time to HCC diagnosis and the levels of the 11 FAs described above, progressors were separated into progressors who developed HCC within 18 months of follow-up (<18 m) and progressors who developed HCC more than 18 months after enrollment (>18 m). The four SFAs, 20:0, 22:0, 23:0 and 24:0, were significantly lower in progressors compared to non-progressors independent of the time to diagnosis (FIG. 2B-2C). In contrast, no statistical difference in abundance was observed for the four n-3 PUFAs and three n-6-PUFAs between progressors >18 m and non-progressors (FIG. 2B-2C), suggesting that changes in abundance for these specific PUFAs occur close to HCC development.

The relation between the levels of the 11 FAs and time to diagnosis was further confirmed by correlation analysis. There was no significant correlation between SFAs levels and time to HCC. In contrast, levels of 18:3n3 and 18:2n6 negatively correlated with time to diagnosis (r=−0.38; p=0.02 and r=−0.40; p=0.012, respectively) (FIG. 3A) and levels of 22:5n3 and 22:4n6 positively correlated with time to diagnosis (r=0.40, p=0.013 and r=0.37; p=0.024, respectively) (FIG. 3B). The correlations were stronger for 22:5n3/18:3n3 ratio (r=0.44; P=0.006) and 22:4n6/18:2n6 ratio (r=0.42; P=0.008) (FIG. 3C).

4. Performance of the Identified FAs in Predicting HCC Diagnosis During Surveillance in Patients with Cirrhosis

ROC analysis showed that compared to AFP, each of the 11 FAs that we identified, had similar or better performance in discriminating cirrhosis patients who developed HCC (progressors) from cirrhosis non-progressors, with AUCs ranging from 0.67 [95% CI: 0.55-0.78] (P=0.02) for 22:4n6 to 0.77 [95% CI: 0.68-0.87] (P<0.0001) for 20:0 acid (Table 2). Compared to OPN, six of these FAs had similar or better performance in discriminating between progressors and non-progressors (Table 2). The performance of the SFAs with the exception of 23:0, was comparable when progressors were separated into progressors >18 m and progressors <18 m. In contrast, AUCs for individual PUFAs increased from 0.67-0.75 when using all progressors, to 0.69-0.79 when using progressors <18 m. The highest AUC among PUFAs was observed for the n-3 PUFA, 22:5n3, with 0.79 [95% CI: 0.69-0.89]. When using progressors >18 m, ROC AUCs for the PUFAs were not statistically significant (Table 2). Overall, the FAs with the best performance were 20:0, 22:5n3 and 18:2n6.

We then evaluated whether combination of SFAs or n-3 PUFAs with AFP or OPN could improve the performance of the biomarker panel by first calculating correlations among all markers (Supplementary Table S2) When using a cutoff of R>0.5 or <−0.5 and p<0.01, positive correlations were observed between SFAs or between 20:5n3 and 22:5n3 or 22:6n3. Most importantly, there was no correlation between the selected 8 FAs and AFP or OPN. We, therefore, evaluated the performance of combining the 4 long chain SFAs and the 4 n-3 PUFAs with AFP and OPN (Supplementary Table S3). When all progressors were included independently of time to diagnosis, the AUCs ranged from 0.78 to 0.81 with the highest AUCs observed for the combination of 20:0+AFP+OPN (0.81 [95% CI: 0.72-0.89]) (FIG. 4A). Addition of both 20:0 and 22:5n3 to AFP and OPN further improved AUC to 0.83 [0.75-0.91] (FIG. 4C).

TABLE 2
Receiver operating characteristics for selected free FAs. ns: not significant;
AUC, area 437 under the curve; CI, confidence interval; 18 m, 18 months.

| | Non-progressors vs | | Non-progressors vs | | Non-progressors vs | |
|---|---|---|---|---|---|---|
| | Progressors | | Progressors (<18 m) | | Progressors (>18 m) | |
| Biomarker | AUC [95% CI] | p-value | AUC [95% CI] | p-value | AUC [95% CI] | p-value |
| 20:0 | 0.77 [0.68-0.87]^{a} | <0.0001 | 0.79 [0.69-0.89] | <0.0001 | 0.75 [0.61-0.89] | 0.003 |
| 22:0 | 0.74 [0.65-0.84] | <0.0001 | 0.75 [0.64-0.86] | 0.0003 | 0.73 [0.59-0.88] | 0.006 |
| 23:0 | 0.73 [0.62-0.83] | 0.0002 | 0.77 [0.67-0.88] | <0.0001 | | Ns |
| 24:0 | 0.72 [0.61-0.82] | 0.0002 | 0.72 [0.60-0.83] | 0.002 | 0.72 [0.57-0.86] | 0.01 |
| 18:3n3 | 0.71 [0.60-0.82] | 0.005 | 0.75 [0.63-0.87] | 0.003 | | Ns |
| 20:5n3 | 0.70 [0.59-0.81] | 0.0007 | 0.76 [0.65-0.88] | 0.0002 | | Ns |
| 22:5n3 | 0.74 [0.64-0.85] | <0.0001 | 0.79 [0.69-0.89] | <0.0001 | | Ns |
| 22:6n3 | 0.69 [0.57-0.81] | 0.005 | 0.75 [0.62-0.89] | 0.004 | | Ns |
| 18:2n6 | 0.75 [0.64-0.85] | <0.0001 | 0.79 [0.67-0.91] | <0.0001 | | Ns |
| 20:4n6 | 0.69 [0.57-0.81] | 0.002 | 0.73 [0.58-0.87] | 0.0007 | | Ns |
| 22:4n6 | 0.67 [0.55-0.78] | 0.02 | 0.69 [0.57-0.81] | 0.01 | | Ns |

^{a}The highest performances in each comparison are shown in bold.

SUPPLEMENTARY TABLE S2
Correlation between biomarker candidates.

| marker | 20:0 | 22:0 | 23:0 | 24:0 | 18:3n3 | 20:5n3 | 22:5n3 | 22:6n3 | AFP | OPN |
|---|---|---|---|---|---|---|---|---|---|---|
| 20:0 | 1.00 | 0.84 | 0.77 | 0.78 | −0.40 | 0.16 | 0.31 | 0.27 | −0.14 | 0.06 |
| | | (p < 0.001) | (p < 0.001) | (p < 0.001) | (p = 0.01) | (p = 0.32) | (p = 0.05) | (p = 0.09) | (p = 0.40) | (p = 0.73) |
| 22:0 | | 1.00 | 0.78 | 0.91 | −0.44 | 0.09 | 0.21 | 0.13 | −0.10 | −0.08 |
| | | | (p < 0.001) | (p < 0.001) | (p = 0.006) | (p = 0.57) | (p = 0.21) | (p = 0.43) | (p = 0.54) | (p = 0.62) |
| 23:0 | | | 1.00 | 0.74 | −0.35 | 0.37 | 0.51 | 0.31 | −0.16 | −0.26 |
| | | | | (p < 0.001) | (p < 0.001) | (p < 0.001) | (p < 0.001) | (p = 0.001) | (p = 0.11) | (p = 0.009) |
| 24:0 | | | | 1.00 | −0.37 | 0.20 | 0.26 | 0.22 | −0.13 | 0.020 |
| | | | | | (p = 0.02) | (p = 0.22) | (p = 0.11) | (p = 0.18) | (p = 0.44) | (p = 0.91) |
| 18:3n3 | | | | | 1.00 | −0.13 | −0.30 | −0.42 | 0.11 | 0.13 |
| | | | | | | (p = 0.43) | (p = 0.06) | (p = 0.008) | (p = 0.52) | (p = 0.42) |
| 20:5n3 | | | | | | 1.00 | 0.69 | 0.50 | 0.05 | 0.11 |
| | | | | | | | (p < 0.001) | (p = 0.001) | (p = 0.77) | (p = 0.49) |
| 22:5n3 | | | | | | | 1.00 | 0.28 | −0.02 | −0.09 |
| | | | | | | | | (p = 0.07) | (p = 0.92) | (p = 0.61) |
| 22:6n3 | | | | | | | | 1.00 | 0.07 | 0.20 |
| | | | | | | | | | (p = 0.70) | (p = 0.23) |
| AFP | | | | | | | | | 1.00 | 0.07 |
| | | | | | | | | | | (p = 0.71) |
| OPN | | | | | | | | | | 1.00 |

AFP, Alpha-fetoprotein;
OPN, osteopontin

SUPPLEMENTARY TABLE S3
Receiver operating characteristics of combinations
of selected fatty acids with AFP and OPN.

| | Non-progressors | | Non-progressors | |
|---|---|---|---|---|
| | vs Progressors | | vs Progressors (<18 m) | |
| Fatty Acids + | AUC | p- | AUC | p- |
| AFP + OPN | [95% CI] | value | [95% CI] | value |
| 20:0 + AFP + OPN | 0.81 | <0.0001 | 0.84 | <0.0001 |
| | [0.72-0.89] | | [0.75-0.93] | |
| 22:0 + AFP + OPN | 0.79 | <0.0001 | 0.81 | 0.0002 |
| | [0.70-0.88] | | [0.72-0.91] | |
| 23:0 + AFP + OPN | 0.78 | <0.0001 | 0.84 | <0.0001 |
| | [0.69-0.88] | | [0.75-0.92] | |
| 24:0 + AFP + OPN | 0.79 | <0.0001 | 0.82 | 0.0002 |
| | [0.70-0.88] | | [0.73-0.91] | |
| 18:3n3 + AFP + OPN | 0.78 | 0.0003 | 0.81 | 0.0003 |
| | [0.68-0.87] | | [0.72-0.91] | |
| 20:5n3 + AFP + OPN | 0.79 | <0.0001 | 0.85 | <0.0001 |
| | [0.70-0.88] | | [0.76-0.94] | |
| 22:5n3 + AFP + OPN | 0.80 | <0.0001 | 0.86 | <0.0001 |
| | [0.71-0.89] | | [0.77-0.94] | |
| 22:6n3 + AFP + OPN | 0.80 | <0.0001 | 0.86 | <0.0001 |
| | [0.71-0.90] | | [0.75-0.96] | |

AFP, Alpha-fetoprotein; OPN, osteopontin; AUC, area under the curve; CI, confidence interval.

When only progressors with a diagnosis of HCC within 18 months of surveillance were included, the AUCs ranged from 0.81 to 0.86 with the highest AUC observed for the combination of 22:5n3+AFP+OPN (0.86 [95% CI: 0.77-0.94]) (FIG. 4B). Addition of both 20:0 and 22:5n3 to AFP and OPN further improved AUC to 0.87 [95% CI: 0.89-0.95] (FIG. 4D). The 4 markers panel detected pre-HCC within 18 months of diagnosis with 82% sensitivity at 81% specificity.

In this study, we evaluated the ability of AFP, OPN, and selected FAs to predict HCC development in patients with cirrhosis under surveillance. We validated in these study patients, that OPN had better performance than AFP in detecting HCC at preclinical stage and that their combination improved the performance of each marker used individually. We then performed FA profiling and identified 11 FAs that presented at significantly different levels between cirrhotic patients who developed HCC during follow-up and those who did not develop HCC. The abundance of the 4 SFAs (20:0, 22:0, 23:0, and 24:0) was lower in cirrhotic patients who developed HCC. The lower levels of these SFAs were independent of the time to diagnosis. Among them, 20:0 and 24:0 had the best capacity to detect preclinical HCC. We observed that the abundance of the long chain n-3 PUFAs was lower in cirrhotic patients who developed HCC during follow-up. Unlike SFAs, n-3 PUFAs showed a significant correlation with time between marker measurement and HCC diagnosis. Significant differences in n-3 PUFA levels were observed in particular, within 18 months of HCC diagnosis. Overall, the best predictive model was a combination of 20:0, 22:5n3, AFP, and OPN with AUC of 0.87 in patients within 18 months of HCC diagnosis, with a sensitivity of 82% at 81% specificity. Such panel can therefore have utility in surveillance, identifying low risk cirrhotic patients that could be spared from bi-annual visits and high risk cirrhotic patients that would benefit from MRI or CT instead of ultrasound to detect very early HCCs.

### Example 2—Detection of HCC Biomarker in Samples from Hispanic Patients with Advanced Liver Fibrosis

This experiment was devised to determine whether circulating FFAs are associated with advanced liver fibrosis and HCC in the Hispanic population of South Texas and could serve as risk prediction biomarkers or therapeutic targets.

A. Materials and Methods

Study participants. The study includes 116 participants from the CCHC (Fisher-Hoch, S. P., et al., Socioeconomic status and prevalence of obesity and diabetes in a Mexican American community Cameron County, Texas, 2004-2007. Preventing chronic disease. 2010; 7(3):A53). Vibration-controlled transient elastography (VCTE—Fibroscan® 502 Touch or Fibroscan® 530 Compact, Echosens) with automatic probe selection, was used to assess liver steatosis measured by controlled attenuation parameter (CAP) and liver fibrosis measured by stiffness (LSM) in kiloPascals (kPa). The following criteria were used: CAP >281 for steatosis and LSM≥8.8 kPa for advanced liver fibrosis. Among the 116 participants, 39 were selected for having advanced liver fibrosis and the other 77 were randomly selected from the CCHC. The baseline clinical characteristics for the cirrhotic study participants is shown in Supplementary Table S1. The study also included 15 Hispanic patients with advanced liver fibrosis and HCC, enrolled at the Doctors Hospital of Renaissance, and 56 first- and second-degree relatives of Hispanic patients with HCC (Supplementary Table S2). All study participants underwent a comprehensive clinical exam, detailed health history and demographic interview. Hepatitis C virus (HCV) antibodies and hepatitis B virus (HBV) surface antigen were assayed using Ortho HCV and Abnova HBsAg ELISAs. Subjects positive for HCV or HBV were excluded from the study. Fasting blood samples were analyzed for metabolic and lipid panels. The following criteria were used: obesity (BMI ≥30), pre-diabetes (no history of diabetic medication, plus either fasting blood glucose of 100-125 mg/dl or HbA1c of 5.7-6.4%), diabetes (fasting blood glucose ≥126 mg/dl, HbA1c ≥6.5% or history of diabetic medication), abnormal aspartate aminotransferase (AST) (>33 U/L), abnormal alanine aminotransferase (ALT) (>40 U/L for males; >31 U/L for females), heavy drinking (weekly consumption of >14 drinks for men and >7 for women), moderate drinking (non-zero weekly consumption that did not reach criteria for heavy drinking), former smoking (lifetime cigarette consumption ≥100, plus no smoking at the time of survey), current smoking (lifetime cigarette consumption ≥100, plus smoking at the time of survey) (Lim, K., et al., Omega-3 polyunsaturated fatty acids inhibit hepatocellular carcinoma cell growth through blocking beta-catenin and cyclooxygenase-2. Mol Cancer Ther. 2009; 8(11):3046-55).

SUPPLEMENTARY TABLE S1
Demographic and clinical characteristics of 116 CCHC subjects (77 without advanced liver fibrosis and
39 with advanced liver fibrosis) and of 15 Mexican American patients with advanced fibrosis and HCC.

| | No Advanced | | Advanced | | | | | |
|---|---|---|---|---|---|---|---|---|
| | Fibrosis | | Fibrosis | | HCC | | | |
| | (n=77) | | (n=39) | | (n=15) | | p* | p** |
| Male | 36 | (46.8%) | 12 | (30.8%) | 13 | (86.7%) | 0.114 | 0.001 |
| Age | 56.4 (18.0-78.0) - 59.0 | | 55.7 (23.0-80.0) - 59.0 | | 65.2 (41.0-80.0) - 65.0 | | 0.817 | 0.026 |
| BMI | 31.8 (22.8-54.0) - 30.7 | | 35.1 (25.9-57.1) - 31.4 | | 36.4 (22.2-108.2) - 29.4 | | 0.029 | 0.819 |
| Obese (BMI ≥30) | 46 | (59.7%) | 25 | (64.1%) | 7 | (46.7%) | 0.691 | 0.355 |
| Hb1Ac (%) | 7.0 (4.6-12.0) - 6.2 | | 6.8 (4.0-12.7) - 6.2 | | 6.7 (4.4-10.7) - 5.8 | | 0.638 | 0.917 |
| Diabetic groups | | | | | | | 0.544 | 0.334 |
| Normal | 13 | (16.9%) | 9 | (23.1%) | 3 | (20.0%) | | |
| Prediabetic | 31 | (40.3%) | 12 | (30.8%) | 2 | (13.3%) | | |
| Diabetic | 33 | (42.9%) | 18 | (46.2%) | 10 | (66.7%) | | |
| Waist circumference (cm) | 106.1 (81-142) - 105 | | 115.1 (89-153) - 106 | | 105.4 (85-141) - 102 | | 0.009 | 0.073 |
| CAP (dB/m) | 291.5 (100-400) - 300 | | 297.3 (151-400) - 301 | | 253.4 (104-400) - 251 | | 0.635 | 0.022 |
| Steatosis status | | | | | | | >0.999 | 0.017 |
| Steatosis (CAP >281 dB/m) | 48 | (62.3%) | 25 | (64.1%) | 4 | (26.7%) | | |
| LSM (kPa) | 5.2 (1.7-8.7) - 4.8 | | 21.9 (8.8-75.0) - 17.3 | | 34.4 (9.6-75.0) - 27.3 | | <0.001 | 0.032 |
| Drinks per week | 2.2 (0.0-33.0) - 0.0 | | 1.5 (0.0-18.3) - 0.0 | | 0.2 (0.0-2.0) - 0.0 | | 0.502 | 0.055 |
| Drinking status | | | | | | | 0.834 | 0.377 |
| Never | 50 | (64.9%) | 27 | (69.2%) | 13 | (86.7%) | | |
| Moderate | 21 | (27.3%) | 10 | (25.6%) | 2 | (13.3%) | | |
| Heavy | 6 | (7.8%) | 2 | (5.1%) | 0 | (0.0%) | | |
| Smoking status | | | | | | | 0.238 | 0.01 |
| Never | 44 | (57.1%) | 28 | (71.8%) | 5 | (33.3%) | | |
| Former | 25 | (32.5%) | 7 | (17.9%) | 9 | (60.0%) | | |
| Current | 8 | (10.4%) | 4 | (10.3%) | 1 | (6.7%) | | |
| Blood tests | | | | | | | | |
| AST (U/L) | 21.5 (7.0-55.0) - 20.0 | | 43.7 (13.0-145.0) - 39.0 | | 49.3 (24.0-98.0) - 51.0 | | <0.001 | 0.496 |
| Abnormal AST (>33 U/L) | 6 | (7.8%) | 22 | (56.4%) | 9 | (60.0%) | <0.001 | >0.999 |
| ALT (U/L) | 33.4 (0-109) - 29 | | 53.1 (13-240) - 34 | | 38.1 (11-77) - 34 | | 0.019 | 0.114 |
| Abnormal ALT (>40 U/L , | 27 | (35.1%) | 21 | (53.8%) | 4 | (26.7%) | 0.072 | 0.127 |
| male >31 U/L female) | | | | | | | | |

| | | | | | |
|---|---|---|---|---|---|
| Albumin (g/dL) | 3.9 (3.2-4.6) - 4.0 | 3.7 (2.5-4.7) - 3.8 | 3.2 (2.0-4.1) - 3.2 | 0.008 | <0.001 |
| Alkaline phosphatase (U/L) | 85.2 (29-155) - 83 | 115.6 (56-184) - 103 | 167.2 (54-371) - 159 | <0.001 | 0.025 |
| FBG (mg/dL) | 126.9 (64-339) - 104 | 119.4 (70-327) - 106 | 126.3 (72-268) - 104 | 0.487 | 0.669 |
| Insulin (mU/L) | 12.5 (0.1-48.6) - 10.1 | 16.2 (1.7-49.4) - 14.3 | 11.9 (1.3-27.3) - 10.3 | 0.06 | 0.178 |
| Insulin resistance (HOMA) | 3.9 (0.0-24.2) - 2.9 | 4.6 (0.9-15.6) - 3.8 | 4.0 (0.4-12.0) - 2.5 | 0.407 | 0.608 |
| Platelets (×10⁹/L) | 238.4 (119-430) - 232 | 180.6 (28-339) - 186 | 129.6 (63-190) - 113 | <0.001 | 0.008 |
| Triglyceride (mg/dL) | 172.9 (54-1429) - 140 | 136.5 (46-486) - 111 | 118.3 (60-177) - 124 | 0.12 | 0.265 |
| Total cholesterol (mg/dL) | 181.8 (104-295) - 183 | 170.3 (92-290) - 156 | 157.6 (107-218) - 158 | 0.169 | 0.314 |
| HDL (mg/dl) | 46.5 (17-84) - 46 | 49.7 (25-80) - 48 | 45.1 (21-97) - 40 | 0.226 | 0.346 |
| LDL (mg/dL) | 102.2 (36-193) - 101 | 92.7 (28-180) - 87 | 88.9 (33-125) - 98 | 0.160 | 0.722 |

BMI, body mass index; HbAlc, hemoglobin Alc; CAP, controlled attenuation parameter measured by Fibroscan; LSM, liver stiffness measurement measured by Fibroscan; AST, aspartate aminotransferase; ALT, alanine aminotransferase; FBG, fasting blood glucose; HDL, high density lipoprotein; LDL, low density lipoprotein.
Data are presented as Mean (range) - median or frequency (%).
*comparison between subjects without advanced fibrosis and subjects with advanced fibrosis;
**comparison between subjects with advanced fibrosis and subjects with HCC

SUPPLEMENTARY TABLE S2
Demographic and clinical parameters of 117 CCHC subjects without liver fibrosis:
56 first- and second-degree relatives of patients with HCC and 61 without family history of HCC.

| | Relatives | | Without family | | |
|---|---|---|---|---|---|
| | (n = 56) | | history (n = 61) | | p |
| Male | 20 | (35.7%) | 27 | (44.3%) | 0.450 |
| Age | 43.8 (18.0-74.0) -44.5 | | 57.6 (18.0-78.0) -59.0 | | <0.001 |
| BMI | 30.8 (22.2-44.8) -30.7 | | 31.3 (24.1-41.2) -30.7 | | 0.569 |
| Obese (BMI ≥30) | 31 | (55.4%) | 37 | (60.7%) | 0.579 |
| Hb1Ac (%) | 6.2 (4.6-13.5) -5.7 | | 7.0 (4.6-12.0) -6.2 | | 0.023 |
| Diabetic groups | | | | | <0.001 |
| Normal | 24 | (42.9%) | 8 | (13.1%) | |
| Prediabetic | 22 | (39.3%) | 27 | (44.3%) | |
| Diabetic | 10 | (17.9%) | 26 | (42.6%) | |
| Waist circumference (cm) | 99.8 (47-130) -99 | | 105.8 (81-140) -105 | | 0.015 |
| CAP (dB/m) | 270.3 (100-390) -278 | | 290.8 (100-400) -302 | | 0.082 |
| Steatosis status | | | | | 0.197 |
| Steatosis (CAP >281 dB/m) | 27 | (48.2%) | 37 | (60.7%) | |
| LSM (kPa) | 4.5 (1.7-7.0) -4.4 | | 4.5 (1.7-7.0) -4.4 | | 0.956 |
| Drinks per week | 1.5 (0-30) -0 | | 1.9 (0-33) -0 | | 0.697 |
| Drinking status | | | | | 0.138 |
| Never | 32 | (57.1%) | 41 | (67.2%) | |
| Moderate | 23 | (41.1%) | 16 | (26.2%) | |
| Heavy | 1 | (1.8%) | 4 | (6.6%) | |
| Smoking status | | | | | 0.042 |
| Never | 43 | (76.8%) | 34 | (55.7%) | |
| Former | 8 | (14.3%) | 20 | (32.8%) | |
| Current | 5 | (8.9%) | 7 | (11.5%) | |
| Blood tests | | | | | |
| AST (U/L) | 22.1 (9.0-56.0) -18.5 | | 20.8 (7.0-46.0) -19.0 | | 0.496 |
| Abnormal AST (>33 U/L) | 10 | (17.9%) | 4 | (6.6%) | 0.086 |
| ALT (U/L) | 36.0 (11.0-127.0) -27.0 | | 32.3 (0.0-109.0) -27.0 | | 0.368 |
| Abnormal ALT (>40 U/L male, >31 U/L female) | 21 | (37.5%) | 19 | (31.1%) | 0.559 |

| | | | |
|---|---|---|---|
| Albumin (g/dL) | 4.0 (3.5-4.8) -4.0 | 4.0 (3.2-4.6) -4.0 | 0.086 |
| Alkaline phosphatase (U/L) | 96.3 (39-226) -87 | 84.8 (49-155) -82 | 0.023 |
| FBG (mg/dL) | 111.6 (68-392) -93 | 124.2 (64-280) -104 | 0.221 |
| Insulin (mU/L) | 10.2 (2.6-38.9) -7.6 | 12.0 (0.1-48.6) -10.0 | 0.261 |
| Insulin resistance (HOMA) | 2.8 (0.5-13.7) -1.8 | 3.7 (0.0-24.2) -2.8 | 0.134 |
| Platelets (x109/L) | 261.3 (154-441) -252 | 236.8 (119-346) -231 | 0.020 |
| Triglyceride (mg/dL) | 153.0 (45-525) -122 | 159.9 (62-550) -141 | 0.683 |
| Total cholesterol (mg/dL) | 181.2 (105-312) -177 | 182.9 (104-283) -183 | 0.821 |
| HDL (mg/dL) | 47.2 (29-71) -46 | 47.3 (22-84) -46 | 0.965 |
| LDL (mg/dL) | 101.4 (7.7-223) -102 | 103.7 (36-193) -103 | 0.727 |

BMI: body mass index; HbAlc: hemoglobin Alc; CAP: controlled attenuation parameter measured by Fibroscan; LSM: liver stiffness measurement measured by Fibroscan; AST: aspartate aminotransferase; ALT: alanine aminotransferase; FBG: fasting blood glucose.
Data are presented as Mean (range) - median or frequency (%).

Absolute quantification of plasma FFAs. FFA profiling was performed at the Metabolomics Core at MD Anderson Cancer Center, using a chemical derivatization approach (Li, X., Franke, A. A. Improved LC-MS method for the determination of fatty acids in red blood cells by LC-orbitrap MS. Anal. Chem. 2011; 83(8):3192-8) and blinded to clinical data. Internal standard mixture consisted of 12.5 μg/mL of (1, 2, 3, 4, 5, 6-¹³C₆) 22:0 and 25 μg/mL ¹³C-labeled 14:0, 16:1n7c, 16:0, 17:0, 18:2n6, 18:1n9c, 18:1n9t, and 18:0 in ethanol (Cambridge Isotope Laboratories, Tewksbury, MA, USA). To vials containing 20 μL of plasma, 32 μL of internal standard mixture and extraction solvent (1 mL) were added. Following centrifugation at 4,122 g at 4° C. for 10 min, the supernatants were transferred to 2 mL vials with Teflon caps and dried using a centrifugal vacuum concentrator. Extracted FFAs were converted to acyl chloride intermediates by treatment with 2004, of two molar oxalyl chloride in dichloromethane at 65° C. for 5 min. The solutions were then dried and samples were derivatized by adding 150 μL of 1% (v/v) 3-picolylamine in acetonitrile. Finally, the solutions were dried and stored at −80° C. Derivatization products were reconstituted in 100 μL ethanol, transferred to auto-sampler vials, dried, and then reconstituted in 154, ethanol. Injection volume was 5 μL. Mobile phase A (MPA) was 0.1% formic acid in water, and mobile phase B (MPB) was 0.1% formic acid in acetonitrile. Chromatographic method included a Thermo Fisher Scientific Accucore C30 column (2.6 μm, 150×2.1 mm) and the following gradient elution: 0-5 min, 65% MPB; 5-5.1 min, 65-90% MPB; 5.1-55 min, 90% MPB; 55-55.1 min, 90-65% MPB; 55.1-60 min, 65% MPB. A Thermo Fisher Scientific Orbitrap Fusion Tribrid mass spectrometer with heated electrospray ionization source was operated in data dependent acquisition mode with a scan range of 150-550 m/z.

SNP genotyping. Patatin-like phospholipase domain-containing protein 3 (PNPLA3) rs738409 and transmembrane 6 superfamily member 2 (TM6SF2) rs58542926 were genotyped by TaqMan 5′-nuclease assays using predesigned TaqMan probes (Applied Biosystems, Foster City, CA), on a ViiA7 Real time PCR system (Applied Biosystems, Foster City, CA).

Statistics. Demographic and clinical parameters were compared between subject groups using two-tailed student's t-test for continuous variables and Fisher tests for categorical variables. Differences in FFA levels were tested using Mann-Whitney Utest or Kruskal-Wallis test. Logistic regression was performed to predict sample status (control vs. case) based on continuous biomarker levels. For each logistic regression model, the area under the receiver operating characteristic (ROC) curve (AUC) was calculated. A likelihood ratio test was conducted to compare models. Correlations between FFA levels and LSMs or diagnostic markers for advanced liver fibrosis were assessed using Spearman correlation analysis. Redundancy analysis (RDA) was performed to evaluate effects of clinical or demographic parameters on FFA profiles. RDA was performed using the RDA function in the Vegan package for R. Log 10-transformed abundances of FFAs were the response variables; log 10-transformed BMI, waist circumference, advanced fibrosis and steatosis were the explanatory variables. Analysis of variance-like, permutation-based tests were used to assess the significance of the model and of each constrained axis, as well as the marginal effects of each explanatory variable. All results are given as two-tailed p-values, and p<0.05 was considered statistically significant. Logistic regression was performed using SPSS to estimate the odds ratio (OR) or adjusted odds ratio (AOR) and 95% confidence interval (CI) for association of FFA with disease status or family history.

B. Results

1. Changes in Plasma FFA Concentrations Associated with Advanced Liver Fibrosis in Hispanics of South Texas

VCTE-Fibroscan was used to measure liver fibrosis and steatosis in subjects from the CCHC, a population-based cohort of Hispanics in South Texas (Fisher-Hoch, S. P., et al., 2010, supra). We randomly selected 39 CCHC subjects with advanced liver fibrosis (LSM≥8.8 kPa), with an average of 21.9 [8.8-75.0] kPa. We also randomly selected 77 CCHC subjects without advanced liver fibrosis (average of 5.2 [1.7-8.7] kPa). Subjects with HCV or HBV were excluded from the study. Subjects with advanced liver fibrosis were more likely to have elevated AST levels (56.4% vs 7.8%, p<0.001), lower platelet counts (180.6×10⁹/L vs 238.4×10⁹/L, p<0.001) and higher alkaline phosphatase levels (115.6 U/L vs 85.2 U/L, p<0.001). Subjects with advanced liver fibrosis also had higher waist circumference (115.1 cm vs 106.1 cm, p=0.009) and BMI (35.1 vs 31.8, p=0.029). Importantly, there was no difference in gender, age, diabetes, steatosis, alcohol consumption and smoking, between the two groups. Demographic and clinical parameters of the 116 CCHC study participants are described in Supplementary Table S1.

A total of 45 FFAs [14 saturated fatty acids (SFAs), 13 monounsaturated fatty acids (MUFAs) and 18 polyunsaturated fatty acids (PUFAs)] were quantified by mass spectrometry in plasma of the 116 study participants. We compared the absolute concentrations of the 45 FFAs between the 39 subjects with advanced liver fibrosis and the 77 subjects without advanced fibrosis. Subjects with advanced fibrosis had significantly lower levels of odd chain SFAs (45.0 μM vs 75.0 μM, fold change (FC)=−1.7, p<0.001), very long chain (VLC) even chain SFAs (176.604 vs 322.5 μM, FC=−1.8, p<0.001), VLC n-3 PUFAs (73.9 μM vs 160.0 μM, FC=−2.2, p<0.001) and VLC n-6 PUFAs (57.2 μM vs 116.3 μM, FC=−2.0, p<0.001) (FIG. 5A). Individual odd chain SFAs included 17:0 (10.1 μM vs 15.5 μM, FC=−1.5, p=0.008), 19:0 (0.4 μM vs 1.0 μM, FC=−2.3, p<0.001), 21:0 (2.1 μM vs 4.7 μM, FC=−2.2, p<0.001), 23:0 (25.4 μM vs 47.0 μM, FC=−1.8, p<0.001) and 25:0 (0.7 μM vs 1.2 μM, FC=−1.9, p<0.001) (FIG. 5B). Individual VLC SFAs included 20:0 (28.9 μM vs 54.6 μM, FC=−1.9, p<0.001), 22:0 (102.7 μM vs 179.7 mM, FC=−1.7, p<0.001) and 24:0 (45.0 μM vs 88.1 μM, FC=−2.0, p<0.001) (FIG. 5C). Individual VLC PUFAs included for n-3: 20:5n3 (2.9 μM vs 6.904, FC=−2.4, p<0.001), 22:5n3 (4.6 μM vs 10.1 μM, FC=−2.2, p=0.001) and 22:6n3 (66.5 μM vs 143.0 μM, FC=−2.2, p=0.001) (FIG. 5D); and for n-6: 20:4n6 (35.0 μM vs 74.8 μM, FC=−2.1, p<0.001), 22:4n6 (17.6 μM vs 31.9 μM, FC=−1.8, p=0.002) and 24:2n6 (4.5 μM vs 9.6 μM, FC=−2.1, p<0.001) (FIG. 5D).

In logistic regression analysis, low levels (quartile Q1) of odd chain SFAs, VLC even chain SFAs and VLC n-3 PUFAs were strongly associated with advanced liver fibrosis (OR [95% CI): 5.1 [2.1-12.6], p<0.001; 10.1 [3.8-26.4], p<0.001; and 4.2 [1.7-10.1], p=0.001, respectively). Among individual FFAs in these groups, 23:0 and 25:0, 24:0 and 20:5n3 had the strongest associations (OR [95% CI]: 8.0 [3.1-20.3], p<0.001; 8.0 [3.1-20.3], p<0.001; 12.9 [4.8-35.3], p<0.001; and 6.4 [2.6-15.9], p<0.001; respectively).

After adjustment to BMI, age, and gender, low levels (Q1) of odd chain SFAs, VLC even chain SFAs and VLC n-3 PUFAs remained strongly associated with advanced fibrosis (AOR [95% CI]: 5.8 [2.2-15.2], p<0.001; 9.9 [3.5-27.5], p<0.001; and 3.7 [1.5-9.3], p=0.005) with again the strongest associations observed for 25:0, 24:0 and 20:5n3 (AOR [95% CI]: 8.2 [3.0-22.4], p<0.001; 13.7 [4.6-40.6], p<0.001; and 6.2 [2.4-16.1], p<0.001) (FIG. 6).

RDA further confirmed the strong relationship between the presence of advanced liver fibrosis and the abundance of the identified FFAs. In this analysis, the FFAs described in FIG. 5 were used as response variables and the presence of advanced liver fibrosis, presence of steatosis, BMI and waist circumference as explanatory variables. The model was statistically significant (p=0.003) with 9.8% of the FFA profiles explained by the presence of advanced liver fibrosis (p=0.001) (Supplementary Table S3, data plot not shown). No contribution of steatosis, BMI nor waist circumference was observed.

SUPPLEMENTARY TABLE S3
Model analysis.

| | |
|---|---|
| | Variance |
| P | Explained |

Marginal Test

| | | |
|---|---|---|
| Advanced Fibrosis | 0.001 | 9.8% |
| Steatosis | 0.818 | 0.1% |
| Waist Circumference^{a} | 0.551 | 0.4% |
| BMI^{a} | 0.286 | 0.9% |

Anova-like Test

| | |
|---|---|
| Model | 0.003 |
| Axis 1 | 0.001 |
| Axis 2 | 0.994 |

2. VLC n-3 PUFAs Improve the Performance of Current Non-Invasive Biomarkers for the Diagnosis of Advanced Liver Fibrosis in this Population

To evaluate whether addition of selected FFAs could improve the performance of current non-invasive markers for advanced liver fibrosis used clinically, we first performed Spearman correlation analysis of the identified FFAs with aspartate aminotransferase-to-Platelet Ratio Index (APRI), fibrosis 4 index (FIB-4) and NAFLD (Non-Alcoholic Fatty Liver Disease) Fibrosis Score (NFS). While APRI did not correlate with any selected FFAs groups, FIB-4 and NFS negatively correlated with odd chain SFAs (r=−0.21, p=0.026 and −0.29, p=0.001, respectively) and VLC even chain SFAs (r=−0.27, p=0.004 and −0.37, p<0.001, respectively). Since no correlation was observed between VLC n-3 PUFAs and APRI, FIB-4 or NFS, we tested whether VLC n-3 PUFAs could improve the performance of these three non-invasive tests. Among them, APRI had the highest AUC for advanced liver fibrosis (0.84 [95% CI: 0.76-0.92]), followed by FIB-4 (0.78 [95% CI: 0.68-0.87]) and NFS (0.73 [95% CI: 0.63-0.83]). The addition of VLC n3-PUFAs significantly improved their performance, reaching 0.90 [95% CI: 0.85-0.96] for the combination with APRI (p=0.03), 0.84 [95% CI: 0.76-0.91) for the combination with FIB-4 (p=0.12), and 0.78 [95% CI: 0.69-0.87] for the combination with NFS (p=0.08) (FIG. 7). At 85% specificity, the addition of VLC n-3 PUFAs increased the sensitivity of APRI from 70% to 80%. At 85% sensitivity, the addition of VLC n-3 PUFAs increased the specificity of APRI from 54% to 82%.

3. Plasma FFAs Concentrations in Hispanics with HCC in the Context of Advanced Liver Fibrosis

We also investigated whether the concentrations of the identified FFAs further decreased in plasma of Hispanics with HCC in the context of advanced liver fibrosis. We quantified FFAs by mass spectrometry in 15 Hispanics in South Texas diagnosed with HCC. All had advanced liver fibrosis measured by VCTE-Fibroscan. Subjects with HCC were more likely to be male (86.7% vs 30.8%, p=0.001) and to have higher alkaline phosphatase levels (167.2 U/L vs 115.6 U/L, p=0.025), lower albumin levels (3.2 g/dL vs 3.7 g/dL, p<0.001), lower platelet counts (129.6×10⁹/L vs 180.6×10⁹/L, p=0.008) and lower steatosis (CAP=253.4 dB/m vs 297.3 dB/m, p=0.022) (Supplementary Table S1). Abundance of VLC n-3 PUFAs were significantly lower in HCC subjects compared to CCHC subjects with advanced liver fibrosis but no HCC. Individual n-3 PUFAs with significantly lower levels in subjects with HCC included 20:5n3 (0.33 μM vs 2.87 μM, FC=−8.6, p=0.002), 22:5n3 (0.86 μM vs 4.57 μM, FC=−5.3, p=0.020), and 22:6n3 (17.7 μM vs 66.5 μM, FC=−3.8, p=0.030) (FIG. 8A). Abundance of VLC SFAs 24:0 (20.1 μM vs 45.0 μM, FC=−2.2, p=0.034), 23:0 (11.4 μM vs 25.7 μM, FC=−2.3, p=0.024) and 25:0 (0.21 μM vs 0.65 μM, FC=−3.1, p=0.017) were also significantly lower in subjects with HCC compared to subjects with advanced liver fibrosis but no HCC (FIG. 8B). The strongest association was observed for 20:5n3 when evaluating risk of HCC among subjects with advanced liver fibrosis (OR [95% CI]: 5.2 [1.4-19.2], p=0.013; AOR/age and gender [95% CI]: 5.3 [1.0-27.9], p=0.05) or among all study participants (OR [95% CI]: 11.8 [3.4-40.4, p<0.001; AOR/age and gender [95% CI]: 11.2 [2.9-42.3], p<0.001).

4. Potential Genetic Contribution to Low Abundance of VLC n-3 PUFAs and VLC SFAs

In order to determine whether genetic factors could contribute to low abundance of VLC n-3 PUFAs and VLC SFAs associated with advanced liver fibrosis and HCC, we also profiled FFAs in 56 first- and second-degree relatives of Hispanics diagnosed with HCC in South Texas. We confirmed that none of these subjects had liver fibrosis by VCTE-Fibroscan. The relatives of patients with HCC were significantly younger (43.8 vs 57.6, p<0.001) and less likely to be diabetic (17.9% vs 42.6%, p<0.001) than 61 CCHC study participants without liver fibrosis nor family history of HCC (Supplementary Table S2). Remarkably, the VLC n-3 PUFAs and VLC SFAs with low abundance in HCC described above, were also significantly lower in relatives of HCC patients than in CCHC subjects without family history of HCC. These included 20:5n3 (3.6 μM vs 6.7 μM, FC=−1.8, p<0.001), 22:5n3 (6.1 μM vs 9.704, FC=−1.6, p=0.013), 22:6n3 (85.704 vs 141.1 μM, FC=−1.6, p=0.005) (FIG. 9A) as well as 24:0 (50.504 vs 89.4 μM, FC=−1.8, p<0.001), 23:0 (31.104 vs 47.7 μM, FC=−1.5, p=0.009) and 25:0 (0.6704 vs 1.241M, FC=−1.9, p=0.001) (FIG. 9B). In logistic regression analysis, low concentrations (Q1) of 20:5n3 and 24:0 had the strongest associations with family history of HCC (OR [95% CI]: 9.0 [3.1-26.0], p<0.001 and 3.5 [1.4-8.5], p=0.006). After adjustment by age, gender and diabetes, the association with family history of HCC remained strong for both 20:5n3 and 24:0 (AOR [95% CI]: 7.5 [2.3-24.3], p=0.001 and 4.5 [1.6-12.7], p=0.005) (FIG. 9C).

We then evaluated whether known SNPs associated with liver fibrosis and HCC were associated with low concentrations of VLC SFAs and VLC n-3 PUFAs. Polymorphisms in PNPLA3 and TM6SF2 have been previously associated with liver fibrosis and HCC. We genotyped PNPLA3 rs738409 and TM6SF2 rs58542926 in over 900 CCHC subjects as well as in all study participants. As anticipated, the frequency of PNPLA3 rs738409 homozygous alleles in CCHC (27.8%) was significantly higher than in other populations (4.0% in Caucasians and 9.3% in all) and similar to reported frequency in Hispanics in California (34.4%) (FIG. 10A). The frequency of TM6SF2 rs58542926 heterozygous alleles in CCHC (7.6%) was similar to other populations (12.1-16.2%) (FIG. 10A). In our study participants, the frequency of PNPLA3 rs738409 homozygous alleles was 31%, 39% and 40% in subjects without advanced liver fibrosis, with advanced fibrosis or with HCC, respectively (FIG. 10B). The frequency of TM6SF2 rs58542926 heterozygous alleles significantly increased from subjects without advanced fibrosis, to subjects with advanced fibrosis and to subjects with HCC (3%, 16% and 27%, p=0.016) (FIG. 10B). In logistic regression analysis, presence of TM6SF2 rs58542926 heterozygous alleles was associated with increased risk for advanced liver fibrosis (OR [95% CI]: 3.3 [1.0-10.2], p=0.042; AOR/age and gender [95% CI]: 3.2 [1.0-10.1], p=0.045) and HCC (OR [95% CI]: 3.4 [0.9-12.4], p=0.067; AOR [95% CI]: 4.9 [1.0-23.9], p=0.050). Lower concentrations of VLC SFAs, 24:0 in particular, and of VLC n-3 PUFAs were observed in subjects with PNPLA3 variants (FIG. 10C). In contrast, no difference was observed with TM6SF2 variants (FIG. 10C).

In this study, we first observed decreased levels of odd chain SFAs, VLC SFAs, VLC n-3 PUFAs and VLC n-6 PUFAs with advanced liver fibrosis in Hispanics from South Texas. We further showed that VLC n-3 PUFAs could have utility in the diagnosis of advanced fibrosis in this population. Indeed, VLC n-3 PUFAs significantly improved the performance of the non-invasive markers for the diagnosis of advanced fibrosis, APRI, FIB-4 and NF S. Next, we showed that levels of VLC n-3 PUFAs n3 and VLC SFAs further decreased during progression from advanced fibrosis to HCC in Hispanics in South Texas. In summary, we identified FFAs that may play pivotal roles in liver fibrosis progression and HCC development in Hispanics in South Texas. Among them, 24:0 and VLC n-3 PUFAs showed the strongest association with advanced liver fibrosis and HCC, and their levels may be affected by genetic polymorphisms, including in PNPLA3. Remarkably, the addition of VLC n-3 PUFAs to APRI strongly improved the diagnostic performance of APRI for advanced fibrosis.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

All publications and abstracts cited above are incorporated herein by reference in their entirety. It should be understood that the foregoing relates only to preferred embodiments of the present invention and that numerous modifications or alteration may be made therein without departing from the spirit and the scope of the present invention as defined in the following claims.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”. Whenever a range is given in the specification, for example, a temperature range, a time range, or a composition range, all intermediate ranges and subranges, as well as all individual values included in the ranges given are intended to be included in the disclosure.

What is claimed:

1. A method of detecting hepatocellular carcinoma biomarkers in a sample, comprising detecting in the sample two or more hepatocellular carcinoma biomarkers,
wherein the two or more hepatocellular carcinoma biomarkers comprise two or more of Tetracosanoic Acid, Heptadecanoic Acid, Eicosapentaenoic Acid, or Docosapentaenoic Acid, and
wherein the sample is a sample of a bodily fluid obtained from a human subject.

2. The method of claim 1, wherein the hepatocellular carcinoma biomarkers further comprise a hepatocellular carcinoma biomarker selected from the group consisting of 22:6n3-Docosahexaenoic acid, 20:0-Eicosanoic acid, 23:0 Tricosanoic acid and 25:0-Pentacosanoic acid.

3. The method of claim 1, wherein the hepatocellular carcinoma biomarkers further comprise osteopontin or alpha-fetoprotein or both.

4. The method of claim 1, wherein the subject is a patient previously diagnosed with advanced liver cirrhosis.

5. The method of claim 1, wherein the subject is a patient previously diagnosed with liver cirrhosis.

6. The method of claim 6, wherein the liver cirrhosis is advanced liver cirrhosis.

7. The method of claim 1, wherein the detecting comprises:
detection by gas chromatography, mass spectroscopy, gas chromatography-mass spectrometry, or liquid chromatograph-mass spectrometry.

8. The method of claim 7, wherein the mass spectrometry is ultra-high resolution mass spectrometry.

9. The method of claim 1, wherein the bodily fluid is selected from the group consisting of blood, sera, and plasma.

10. The method of claim 1, wherein the bodily fluid is plasma.

11. The method of claim 1, wherein the detecting comprises determining concentrations of the two or more biomarkers in the sample.

12. The method of claim 11, further comprising comparing the levels of the two or more hepatocellular carcinoma biomarkers in the sample to a control level, wherein the control level corresponds to the concentration of the two or more hepatocellular carcinoma biomarkers in a sample from a healthy individual without hepatocellular carcinoma.

13. The method of claim 1, wherein the subject is a subject undergoing treatment for hepatocellular carcinoma or advanced liver fibrosis, and wherein the sample comprises at least two samples obtained at different time points during the treatment.

14. The method of claim 13, wherein at least one of the different time points is a time point prior to start of the treatment.

15. The method of claim 13, wherein the treatment comprises one or more of a drug treatment, a radiation treatment, or a surgical treatment.

16. The method of claim 1, further comprising treating the patient with an anti-hepatocellular carcinoma treatment.

17. The method of claim 16, wherein the treatment comprises one or more of a drug treatment, a radiation treatment or a surgical treatment.

18. A method of generating a report containing information on results of detection of hepatocellular carcinoma biomarkers in a sample, comprising:
detecting in the sample two or more hepatocellular carcinoma biomarkers; and,
generating the report,
wherein the two or more fatty acid hepatocellular carcinoma biomarkers are two or more of Tetracosanoic Acid, Heptadecanoic Acid, Eicosapentaenoic Acid, or Docosapentaenoic Acid,
wherein the sample is a sample of a bodily fluid obtained from a subject,
and wherein the report is useful for diagnosing hepatocellular carcinoma in the subject.

19. The method of claim 18, wherein the hepatocellular carcinoma biomarkers further comprise a hepatocellular carcinoma biomarker selected from the group consisting of 22:6n3-Docosahexaenoic acid, 20:0-Eicosanoic acid, 23:0 Tricosanoic acid and 25:0-Pentacosanoic acid.

20. A system for detecting hepatocellular carcinoma biomarkers in a sample, comprising a station for analyzing the sample by gas chromatography, mass spectrometry, gas chromatography-mass spectrometry, or liquid chromatography-mass spectrometry to detect two or more hepatocellular carcinoma biomarkers in the sample,
wherein the two or more fatty acid hepatocellular carcinoma biomarkers are two or more of Tetracosanoic Acid, Heptadecanoic Acid, Eicosapentaenoic Acid, or Docosapentaenoic Acid, and
wherein the sample is a sample of a bodily fluid obtained from a subject.

21. The system of claim 20, wherein the hepatocellular carcinoma biomarkers further comprise a hepatocellular carcinoma biomarker selected from the group consisting of 22:6n3-Docosahexaenoic acid, 20:0-Eicosanoic acid, 23:0 Tricosanoic acid and 25:0-Pentacosanoic acid.

22. The system of claim 20, further comprising a station for generating a report containing information on results of the analyzing.

23. A method for enhancing the detection of advanced liver fibrosis in a sample comprising detecting advanced liver fibrosis by a standard, non-invasive detection method in combination with detection of one or more hepatocellular biomarkers,
wherein the standard detection method for advanced liver fibrosis is selected from the group consisting of aspartate aminotransferase to platelet ratio index (APRI), fibrosis-4 index (FIB-4), and NAFLD (Non-Alcoholic Fatty Liver Disease) Fibrosis Score (NFS),
wherein the two or more hepatocellular carcinoma biomarkers comprise two or more of Tetracosanoic Acid, Heptadecanoic Acid, Eicosapentaenoic Acid, or Docosapentaenoic Acid,
optionally wherein the hepatocellular carcinoma biomarkers further comprise a hepatocellular carcinoma biomarker selected from the group consisting of 22:6n3-Docosahexaenoic acid, 20:0-Eicosanoic acid, 23:0 Tricosanoic acid and 25:0-Pentacosanoic acid, and
wherein the sample is a sample of a bodily fluid obtained from a human subject.
